(19) United States
(12) Patent Application Publication
(10) Pub. No.: US 2023/0404921 A1
(43) Pub. Date: Dec. 21, 2023

(54) LYOTROPIC LIQUID CRYSTALLINE PHASE PARTICLE

(71) Applicant: ROYAL MELBOURNE INSTITUTE OF TECHNOLOGY, Melbourne (AU)

(72) Inventors: Calum DRUMMOND, Melbourne (AU); Brendan Paul DYETT, Melbourne (AU); Charlotte CONN, Melbourne (AU); Haitao YU, Melbourne (AU)

(21) Appl. No.: 18/250,942

(22) PCT Filed: Oct. 29, 2021
(86) PCT No.: PCT/AU2021/051277

§ 371 Date: Apr. 27, 2023

(30) Foreign Application Priority Data
Oct. 30, 2020 (AU) 2020903958

(51) Int. Cl. A61K 9/127; A61K 47/14; A61K 47/10; A61K 31/4196; A61K 31/7048; A61K 31/496; A61K 31/4409; A61K 31/133; A61K 31/7036; A61K 31/4965; A61K 31/43; A61K 38/14; A61K 31/407; A61P 31/04; A61K 31/431

(52) U.S. Cl. CPC A61K 9/1271; A61K 47/14; A61K 47/10; A61K 31/4196; A61K 31/7048; A61K 31/496; A61K 31/4409; A61K 31/133; A61K 31/7036; A61K 31/4965; A61K 31/43; A61K 38/14; A61K 31/407; A61P 31/04; A61K 31/431

(57) ABSTRACT

The present disclosure provides for a non-lamellar lyotropic liquid crystalline phase particle which is useful for the delivery of active agents to treat gram-negative bacterial and/or fungal infections. The non-lamellar lyotropic liquid crystalline phase particle is shown to provide benefits in delivery of the active agents through a fusion event with the gram-negative bacteria outer membrane or fungi outer layer.

### FIELD OF THE INVENTION

The invention relates to the field of medical treatment and diagnosis of disease. More particularly, this invention relates to a non-lamellar lyotropic liquid crystalline phase particle, which is a carrier particle for an active agent and uses thereof.

### BACKGROUND TO THE INVENTION

Any reference to background art herein is not to be construed as an admission that such art constitutes common general knowledge in Australia or elsewhere.

The increasing prevalence of antibiotic resistant bacteria has been identified as one of the key health risks to humankind. The resistance has largely been driven by overuse of antibiotics prompting resistant genetic mutations. Fiscal and health complications from resistant bacteria are projected to increase into the future. While new means of antimicrobial treatments are currently under extensive investigation, the development of new antibiotics remains slow. The external plasma membrane of gram-negative bacteria, which includes notorious species such as Escherichia coli and Pseudomonas aeruginosa, provides an additional permeation barrier, rendering them particularly resilient against conventional small molecule antibiotics. Gram-negative bacteria which are resistant to last resort antibiotics have already been reported. For this reason, the World Health Organization has prioritized a particularly urgent need for new treatments against gram-negative bacteria.

Due to continued low discovery rates for new antibiotics, the development of technologies which complement existing treatments may prove critical. Delivery technologies and carrier-controlled internalization may provide a more effective treatment for effectively combating bacteria. Particularly, nanomaterials can both act as carriers for single or multiple therapeutic compounds and potentially act to undermine the bacterial membrane barrier, amplifying antimicrobial outcomes. However, the interaction between nanomaterial carriers and bacteria remains poorly understood.¹

In contrast to mammalian cells, which typically internalize nanomaterials via endocytotic pathways,^{2,3} bacteria rely on the permeability of their cell wall to transport material. The innate complexity of the cell wall is largely responsible for difficulties in the development of effective antibiotics and so carriers appropriate for mammalian cells are rarely also appropriate for delivery to bacterial cells. Lyotropic liquid crystalline mesophase carriers, such as cubic phase lipid nanocarriers (cubosomes) have shown some promise in the delivery of therapeutics to mammalian cell lines by means other than endocytosis^{4,5,6,7}. The mechanism of delivery, and the ensuing reliability and extent of delivery of any encapsulated active, is still unresolved in the existing literature.

Similarly, resistant fungal infections are increasing in prevalence and threaten current health practices. Once in the blood stream, these infections have a fatality rate of approximately 25%. The estimated medical cost is US$3 billion per annum. Antifungal drugs often display high cell toxicity and are poorly water soluble. Similar to bacteria, outer cell wall materials e.g. chitin, of fungi can provide a significant diffusion barrier to antimicrobials.

There is a need to provide additional carriers to deliver antimicrobials, including antibacterials and antifungals, and other biologically active agents. There is a further need for the development of new lyotropic liquid crystalline phase particles, as carriers of such actives, for more effective treatment of microbial infections amongst other medical uses.

### SUMMARY OF INVENTION

According to a first aspect of the invention, there is provided a non-lamellar lyotropic liquid crystalline phase particle comprising one or more fusogenic amphiphilic lipids, and the particle encapsulating an active agent.

In a second aspect, there is provided a pharmaceutical composition comprising a non-lamellar lyotropic liquid crystalline phase particle of the first aspect and a pharmaceutically acceptable carrier, diluent and/or excipient.

In a third aspect, there is provided a method of controlled release of an active agent including the steps of forming a non-lamellar lyotropic liquid crystalline phase particle of the first aspect; and administering the non-lamellar lyotropic liquid crystalline phase particle to a target area.

In a fourth aspect, there is provided a method of forming a non-lamellar lyotropic liquid crystalline phase particle of the first aspect including the steps of: (i) providing one or more fusogenic amphiphilic lipids; and (ii) exposing the one or more fusogenic amphiphilic lipids to a solution in the presence of an active agent.

In a fifth aspect, there is provided a method of treatment or prevention of a disease, disorder or condition including the step of administering a therapeutically effective amount of a non-lamellar lyotropic liquid crystalline phase particle of the first aspect, or the pharmaceutical composition of the second aspect, to a subject in need thereof to thereby treat or prevent the disease, disorder or condition.

In a sixth aspect, there is provided a non-lamellar lyotropic liquid crystalline phase particle of the first aspect, or the pharmaceutical composition of the second aspect, for use in the treatment or prevention of a disease, disorder or condition.

In a seventh aspect, there is provided a use of a non-lamellar lyotropic liquid crystalline phase particle of the first aspect in the manufacture of a medicament for the treatment of a disease, disorder or condition.

In an eighth aspect, there is provided a method of delivering an active agent to a biological target including the step of administering a non-lamellar lyotropic liquid crystalline phase particle of the first aspect.

In a ninth aspect, there is provided a method of diagnosing a disease, disorder or condition in a mammal including the step of administering a non-lamellar lyotropic liquid crystalline phase particle of the first aspect or a composition of the second aspect, wherein the active agent within the non-lamellar lyotropic liquid crystalline phase particle of the first aspect is a labelled active agent, to the mammal or to a biological sample obtained from the mammal to facilitate diagnosis of the disease disorder or condition in the mammal.

The various features and embodiments of the present invention, referred to in individual sections above apply, as appropriate, to other sections, mutatis mutandis.

Consequently, features specified in one section may be combined with features specified in other sections as appropriate.

Further features and advantages of the present invention will become apparent from the following detailed description.

### BRIEF DESCRIPTION OF THE DRAWINGS

In order that the invention may be readily understood and put into practical effect, preferred embodiments will now be described by way of example with reference to the accompanying figures wherein:

FIG. 1 is a series of fluorescence images of bacteria in the absence of fluorescently tagged cubosome particles. Left and right show images recorded at λₑₓ=405 nm and λₑₓ=647 nm, respectively. Scale bar=2 μm.

FIG. 2a-f is a series of images and graphical representations showing cubosome uptake by B. cereus. (a) TIRF snapshots showing the in-situ attachment of a single cubosome to the B. cereus surface. Scale bar=5 μm. (b) Mean squared displacement of attaching cubosome. (c) Snapshots highlighting consecutive attachment of MO cubosomes to surface of B. cereus over approximately 4 hours. (d) Long term transfer of fluorescence from particle to B. cereus over 24 hours. Scale bar=20 μm. (e) Extracted fluorescence intensity with time for individual cubosomes in contact with the B. cereus surface. The data is shown on a log scale within the inset and an indicative slope of −½. (f) Extracted intensity with time for B. cereus.

FIG. 3a-g is a series of images and graphical representations showing cubosome uptake by Staphylococcus Aureus. (a-b) TIRF snapshots highlighting consecutive attachment of MO-DOTAP cubosomes to surface of S. aureus. Scale bar=2 μm. (c) Extracted fluorescence intensity with time for individual cubosomes in contact with S. aureus surface. The data is shown on a log-log scale in the inset, with indicative slopes of −⅓ and −½ shown as the solid and dashed lines, respectively. (d) Fluorescence intensity with time for cluster of S. aureus, peptidoglycan extract and peptidoglycan extract treated with lipoteichoic acid. (e) TIRF snapshots highlighting consecutive attachment of MO cubosomes to surface of S. aureus peptidoglycan extract. Scale bar=5 μm. (f) SEM micrographs of S. aureus peptidoglycan extract in the absence and treatment by cubosomes. Scale bar=1 μm. (g) SEM of fixed S. aureus after treatment by cubosomes. Scale bar=300 nm.

FIG. 3h is a series of TIRF snapshots which show the lack of any cubosomes attaching to surface of S. aureus peptidoglycan and lipoteichoic acid extract. Scale bar=5 μm.

FIG. 4a-m is a series of images showing variation in cubosome uptake by Escherichia coli. (a) TIRF snapshots highlighting consecutive attachment of MO cubosomes to surface of Escherichia coli. Scale bar=2 μm. (b) Consecutive attachment and spread of MO-DOTAP cubosomes to surface of Escherichia coli.

Scale bar=2 μm. (c-d) Snapshots at λₑₓ=647 nm, highlighting the apparent burst of fluorescence across E. coli for MO and MO-DOTAP, respectively. Scale bar=2 μm. (e) Snapshots of rapid interaction of two MO-DOTAP cubosomes. The first frame shows fluorescence from λₑₓ=405 nm and following four frames are λₑₓ=647 nm. Two cubosomes are observed landing at 10 and 20 s. In the following frames the cubosomes are no longer present however a gradient in intensity is shown in the bacterium perimeter. (f-m) SEM micrograph of Escherichia coli treated by MO-cubosomes. Scale bar=400 nm.

FIG. 5a-f is a series of images and graphical representations showing cubosome uptake by Escherichia coli. (a-b) Peak intensity profile across an individual E. coli cell for MO and MO-DOTAP, respectively. (c) Intensity across Escherichia coli cell. Left axis shows the total area intensity. Right axis shows the peak intensity. (d) Peak intensity of individual MO-DOTAP cubosomes overtime in contact with Escherichia coli. The inset includes the data replotted on log scales. The steep and shallow dashed lined indicates a slope of −1 and −⅙, respectively. (e) Single representative plot of an individual cubosome on log scales. (f) Corresponding TIRF snapshots for the individual cubosome plotted in (e). Scale bar=2 μm.

FIG. 6a-b are graphical representations showing Incidents of rapid uptake. (a) Peak intensity of individual cubosomes over time in contact with Escherichia coli. The arrows mark points in time where fluorescence rapidly spread throughout the bacterium. (b) The data plotted on log scales with time in seconds. The steep dashed and shallow dashed lines indicate a slope of −1 and −⅙, respectively.

FIG. 7a-f is a series of images and graphical representations showing (a) TIRF time lapse showing fusion of cubosomes (bright point) with SLB. Scale bar=1.5 μm. (b) Uptake of cubosomes into E. coli demonstrates two clear regimes. The inset images are 10 s apart, showing spread of a nanocarrier (NC). (c) Uptake into S. aureus demonstrates one clear regime. (c-d) TIRF time lapse showing the attachment and internalisation of NCs (bright spots) for E. coli and S. aureus, respectively. Scale bar=2 μm (e) Internalisation of NCs (red) into C. albicans.

FIG. 8 shows formulations of cubosomes containing varying degrees of novobiocin against Pseudomonas aeruginosa. Free antibiotic is shown in plain black line. The concentration on x-axis is the drug concentration in solution. Across formulations the drug concentration is consistent. Increasing 1-3-5% mol Novo reflects an increased drug loading per particle however fewer particles are present with increasing concentration.

FIG. 9 shows formulations of cubosomes containing varying degrees of novobiocin against Escherichia coli. Free antibiotic is shown in plain black line. The concentration on x-axis is the drug concentration in solution. Across formulations the drug concentration is consistent. Increasing 1-3-5% mol Novo reflects an increased drug loading per particle however fewer particles are present with increasing concentration.

FIG. 10 shows the inhibition of Pseudomonas aeruginosa by novobiocin formulations in the presence of varying serum environments. The x-axis is concentration of the protein environment, while the concentration of antibiotic is fixed to 20 ug/ml for each formulation. MO-TAP-3Novo was assessed in HSA, BSA and FBS. The equivalent loading of free antibiotic is shown by black square, circles, and triangles respectively. The dashed line indicates the performance of the MO-TAP-3Novo in the absence of any serum proteins.

FIG. 11 shows formulations of cubosomes containing varying degrees of piperacillin against Pseudomonas aeruginosa. Free antibiotic shown in plain black line.

FIG. 12 shows formulations of cubosomes containing varying degrees of piperacillin against Escherichia coli. Free antibiotic shown in plain black line.

FIG. 13 shows formulations of cubosomes containing varying degrees of meropenem against Pseudomonas aeruginosa. Free antibiotic shown in plain black line.

FIG. 14 shows formulations of cubosomes containing varying degrees of meropenem against Escherichia coli. Free antibiotic shown in plain black line.

FIG. 15 shows formulations of cubosomes containing varying degrees of Clarithromycin against Escherichia coli. Free antibiotic shown in plain black line.

FIG. 16 is a photograph indicating that the clarithromycin formulation is prone to sedimentation/poor dispersion.

FIG. 17 shows formulations of cubosomes containing varying degrees of gentamicin against Pseudomonas aeruginosa. Free antibiotic shown in plain black line.

FIG. 18 shows formulations of cubosomes containing varying degrees of gentamicin against Escherichia coli. Free antibiotic shown in plain black line.

FIG. 19 shows formulations of cubosomes containing dicloxacillin and tazobactam against Escherichia coli.

FIG. 20 shows formulations of cubosomes containing benzyl penicillin against Escherichia coli.

FIG. 21 shows formulations of cubosomes containing varying degrees of rifampicin against Escherichia coli.

FIG. 22 shows CFU counts for varying dosages of rifampicin free in solution (solid bars) and encapsulated in MO-1TAP-3Fus (hashed columns). Minimum inhibitory concentration (90% death) indicated by asterisks. For rifampicin, at the lowest concentration examined (0.05 μg/ml), there was already a 50% reduction in CFUs compared to the free drug. Then, at 0.5 μg/ml, the count was dramatically reduced ˜7-fold. The respective MICs, again noted by * on the plot, were 3-5 μg/ml and 1 μg/ml, for the free and encapsulated Rif, respectively, indicating at least a threefold reduction in the MIC. The free MIC value is in reasonable agreement with reported values for Escherichia coli O157:H7 (˜4 μg/ml).

FIG. 23 shows formulations of cubosomes containing 1% rifampicin with varying degrees of DOPE (increasing curvature) of 0-10-20% against Escherichia coli.

FIG. 24 shows formulations of cubosomes containing 1% rifampicin with varying degrees of DOPE (increasing curvature), as for FIG. 23 following delayed testing, against Escherichia coli.

FIG. 25 shows formulations of cubosomes containing 1% rifampicin, 1% DOTAP (positive charge) with varying degrees of DOPE (increasing curvature), of 10 or 20, against Escherichia coli.

FIG. 26 shows formulations of cubosomes containing 1% rifampicin 70% MO, 30% DOPE (mol %), or 1% rifampicin 60% MO, 40% DOPE (mol %) (increasing curvature with increasing DOPE), and so presenting the hexagonal phase, against Escherichia coli.

FIG. 27 is a second data set of the formulations shown in FIG. 26, against Escherichia coli.

FIG. 28 shows formulations of cubosomes containing 1% rifampicin, 1% DOTAP (positive charge) 70% MO and 30% DOPE or 1% rifampicin, 1% DOTAP (positive charge) 60% MO and 40% DOPE (increasing curvature with increasing DOPE), and so presenting the hexagonal phase, against Escherichia coli.

FIG. 29 is a second data set of the formulations shown in FIG. 28, against Escherichia coli.

FIG. 30 shows the CFU counts for varying dosages of fusidic acid free in solution (solid bars) and encapsulated in MO-1DOTAP-3Fus (hashed columns), against Escherichia coli. Minimum inhibitory concentration (90% death) indicated by asterisks.

FIG. 31 shows the viability of Pseudomonas aeruginosa and Escherichia coli against varying concentrations of lipid nanoparticles without encapsulated active by way of a control.

FIG. 32 shows the MIC determination results of rifampicin formulations and incorporation of DOTAP (positive charge) on Mycobacterium smegmatis.

FIG. 33 shows the cell death achieved with rifampicin formulations and incorporation of DOTAP (positive charge) on Mycobacterium smegmatis.

FIG. 34 shows the MIC determination results of rifampicin formulations and incorporation of DOTAP (positive charge) on Mycobacterium tuberculosis H37Ra.

FIG. 35 shows the cell death achieved with rifampicin formulations and incorporation of DOTAP (positive charge) on Mycobacterium tuberculosis H37Ra.

FIG. 36 shows formulations of cubosomes containing 1% filipin, 0/1% DOTAP (positive charge), 0/10% cholesterol against Candida albicans.

FIG. 37 shows formulations of cubosomes containing 1% amphotericin B, 0/1% DOTAP (positive charge), 0/10% cholesterol against Candida albicans.

FIG. 38 shows images obtained by confocal and SEM for fluconazole alone; lipid particles without fluconazole (control nanoparticles) and fluconazole-loaded lipid nanoparticle. a) confocal images at pH-5.0, b) SEM images at pH-5.0, c) confocal images at pH-7.0, d) SEM images at pH-7.0.

### DETAILED DESCRIPTION

The present invention is predicated, at least in part, on the realisation that certain parameters and components of non-lamellar lyotropic liquid crystalline phase carrier particles may be manipulated, such as the nature of the component fusogenic lipids, internal or average spontaneous curvature, charge, drug loading and micro-rheology, to provide for a carrier particle which is capable of one or more of: (i) improved encapsulation of active agent; (ii) improved fusogenic behaviour with microorganisms; (iii) optimal release profile of active agent; (iv) reduction in steric and/or electrostatic barriers to contact with biological membranes; (v) protection of active from damage or binding which would otherwise occur and inactivate or reduce the activity of said active; (vi) ability to deliver active agents which would not otherwise be able to cross the bacterial membrane; and (vii) improvement in efficacy of the active agent compared with delivery of the free active.

The formation of non-lamellar lyotropic liquid crystalline phase carrier particles which can be tailored to improve delivery of specific active agents and/or to specific biological targets will allow for widespread use within medical applications including detection, targeted treatment, imaging and the like.

Particularly, it is shown herein that non-lamellar lyotropic liquid crystalline phase carrier particles can be designed which are capable of delivering a payload to fungi and mycobacteria and gram-negative bacteria. The design of the particles herein may, particularly though not exclusively, lend themselves to the delivery of antibacterial and antifungal agents through fusion of the particle with the bacterial membrane of gram-negative bacteria or the fungal membrane.

Delivery of actives to fungi and, particularly, to gram-negative bacteria is notoriously challenging. As is well-known, for gram-positive species the bacterial membrane consists of a thick outer layer of peptidoglycan and an inner plasma phospholipid membrane. Conversely, gram-negative species exhibit a relatively thinner peptidoglycan layer sandwiched between two phospholipid membranes with lipopolysaccharides featuring on the outer membrane. The outer membrane present in gram-negative species renders them particularly resilient against antimicrobial compounds and presents a unique challenge for delivery of antimicrobial agents.

Unless defined otherwise, all technical and scientific terms used herein have the same meaning as would be commonly understood by those of ordinary skill in the art to which this invention belongs.

In this patent specification, the terms ‘comprises’, ‘comprising’, ‘includes’, ‘including’, or similar terms are intended to mean a non-exclusive inclusion, such that a method or composition that comprises a list of elements does not include those elements solely, but may well include other elements not listed.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The term “non-lamellar lyotropic liquid crystalline phase particle”, as used herein, refers to a self-assembled particle including liquid crystalline non-lamellar structure, formed from at least one amphiphile to give a two and/or three-dimensional mesophase structure which is capable of carrying an active agent. Non-lamellar lyotropic liquid crystalline phase particles are shown herein to result in superior fusion with, and delivery of active agents through, biological membranes. The terms “lipid carrier”, “non-lamellar lyotropic liquid crystalline phase particle”, “non-lamellar LLC particle”, “particle” and “nanoparticle” are used interchangeably herein.

In embodiments, the term “non-lamellar lyotropic liquid crystalline phase particle” may be used to include cubic, hexagonal and sponge morphologies. While the “sponge phase” or “sponge particles” (L₃) are recognised as not possessing long range order and demonstrating equivalent crystalline periodicity of the inverse bicontinuous cubic phase (Q_{II}), they are often considered as a “melted” Q_{II} cubic phase and so are considered to be included as particles of the first aspect. Therefore, short range order sponge phases are explicitly considered to be within the scope of this term. In embodiments, the term “non-lamellar lyotropic liquid crystalline phase particle” may be used to include one or more phases selected from the group consisting of hexagonal (normal and reversed), cubic (normal discrete, reversed discrete, reversed bicontinuous—including primitive, gyroid and diamond—and reversed discontinuous), and other ‘intermediate phases’ including the ribbon, mesh, or non-cubic ‘sponge’ bicontinuous phases. Preferably, the term is used for cubic and/or hexagonal phases.

The terms “amphiphile”, “amphiphilic” and “amphiphilic lipid”, as used herein refer to compounds which comprise both a hydrophilic and a hydrophobic moiety and may be employed as lipids, fusogenic or otherwise, in formation of the LLC particles described herein. Typically, such compounds will have a hydrophilic head group and a hydrophobic tail. Suitable examples include fatty acids and a range of lipid molecules.

The term “fusogenic”, as referred to herein, refers to compounds, typically lipids, which will, as part of a non-lamellar lyotropic liquid crystalline phase particle, promote or enhance fusion of the particle with a biological membrane, such as a bacterial cell membrane, to thereby facilitate delivery of an active agent.

The term “pharmaceutically acceptable salt”, as used herein, refers to salts of the active agent which are toxicologically safe for systemic or localised administration such as salts prepared from pharmaceutically acceptable non-toxic bases or acids including inorganic or organic bases and inorganic or organic acids. The pharmaceutically acceptable salts may be selected from the group including alkali and alkali earth, ammonium, aluminium, iron, amine, glucosamine, chloride, sulphate, sulphonate, bisulphate, nitrate, citrate, tartrate, bitarate, phosphate, carbonate, bicarbonate, malate, maleate, napsylate, fumarate, succinate, acetate, benzoate, terephthalate, palmoate, piperazine, pectinate and S-methyl methionine salts and the like.

According to a first aspect of the invention, there is provided a lipid carrier which may be a non-lamellar lyotropic liquid crystalline phase particle comprising one or more fusogenic amphiphilic lipids, and the particle encapsulating an active agent.

In embodiments, the lipid carrier which may be a non-lamellar lyotropic liquid crystalline phase particle is formed by the self-assembly of the one or more fusogenic amphiphilic lipids. It will be understood that appropriate amphiphilic lipids will self-assemble when in the presence of an aqueous solution, such as water or an aqueous buffer solution, to form a lyotropic liquid crystalline (LLC) structure displaying a non-lamellar mesophase.

In embodiments, the non-lamellar lyotropic liquid crystalline phase particle comprises at least two fusogenic amphiphilic lipids, optionally at least three or at least four, or at least 5, or at least 6, or at least 7, 8, 9 or 10 or more fusogenic amphiphilic lipids.

In embodiments, the non-lamellar lyotropic liquid crystalline phase particle may consist of, or consist essentially of, one, two, three or four fusogenic amphiphilic lipids.

In embodiments, the non-lamellar lyotropic liquid crystalline phase particle may consist of, or consist essentially of, one, two, three or four amphiphilic lipids, fusogenic or non-fusogenic.

In embodiments, the non-lamellar lyotropic liquid crystalline phase particle is formed by the self-assembly of the one or more fusogenic amphiphilic lipids in the presence of the active agent. It will be appreciated that there may be multiple ways in which the active agent can be attached to, incorporated or encapsulated within the particle and the final approach will depend on the nature of the active agent and the manner in which the particle is to deliver it. For example, in certain embodiments, it may be appropriate to focus on attachment of the active to largely the surface of the particle. Typically, however, the particle will be formed in the presence of the active agent so that the active agent is incorporated within the internal channels and folds of the lipid particle in addition to any incidental surface-bound active agent.

In embodiments, a majority of the active agent is located within the internal channels of the particle of the first aspect. Preferably, at least 50%, 60%, 70%, 80%, 90%, 95%, 96%, 97%, 98% or 99% of the active agent associated with the particle of the first aspect at the time of delivery to a target area is located within the internal channels of the particle of the first aspect.

In embodiments, the non-lamellar lyotropic liquid crystalline phase particle may be a colloidal particle, being one with a particle size of less than 10 micrometers.

In embodiments, the particle size of the particle of the first aspect may be between about 10 micrometers and about 40 nanometers. Preferably, the particle size is between about 5 micrometers and about 50 nanometers, more preferably between about 1 micrometer and about 50 nanometers, even more preferably between about 800 nanometers and about 50 nanometers, still more preferably between about 600 nanometers and about 50 nanometers, even yet more preferably between about 500 nanometers and about 50 nanometers or between about 400 nanometers and about 50 nanometers, or between about 5 micrometers and about 100 nanometers, more preferably between about 1 micrometer and about 100 nanometers, even more preferably between about 800 nanometers and about 100 nanometers, still more preferably between about 600 nanometers and about 100 nanometers, even yet more preferably between about 500 nanometers and about 100 nanometers or between about 400 nanometers and about 100 nanometers. The particles of the first aspect may therefore operate as nanocarriers of the active agent within embodiments of the above particle size ranges.

In embodiments, the non-lamellar lyotropic phase particle has a bulk phase selected from the group consisting of the cubic phase, the hexagonal phase and the sponge phase, including normal and inverse/reverse phases of each as appropriate.

Non-lamellar LLC particle matrices offer a range of advantages compared to their lamellar analogues, such as liposomes. Their lipid composition can render them more fusogenic with the outer membrane of bacteria and, owing to their high internal surface area and amphiphilic nature, non-lamellar LLC particles such as cubosomes have the capacity to encapsulate and release an array of actives. Non-lamellar LLC particle matrices can also protect the structural integrity of the encapsulated active agent from enzymatic degradation.

In embodiments, the non-lamellar lyotropic liquid crystalline phase particle is one selected from the group consisting of hexagonal (normal and reversed), cubic (normal discrete, reversed discrete, reversed bicontinuous—including primitive, gyroid and diamond—and reversed discontinuous), and other ‘intermediate phases’ including the ribbon, mesh, or non-cubic ‘sponge’ bicontinuous phases.

In embodiments, the non-lamellar lyotropic liquid crystalline phase particle is one selected from the group consisting of cubosomes, hexosomes and sponge particles.

In embodiments, the non-lamellar lyotropic liquid crystalline phase particle of the first aspect may be a cubosome or a hexosome.

Preferably, the cubosome is a bicontinuous cubic phase (V₁) or inverse bicontinuous cubic phase (V₂) cubosome. Inverse bicontinuous cubic phase (V₂) cubosomes are particularly preferred. It will be appreciated by a person skilled in the art that V₂ is an umbrella term for the varying cubic phases. V₂ can also be referred as V_{II} or Q_{II}. Within Q_{II} there are Q_{II}^{D} (Pn3m), Q_{II}^{P} (Im3m), Q_{II}^{G}(Ia3d).

Inverse (reverse) phase particles may be preferred as they provide for a complex series of internal channels which can accommodate active agent and which allow for a better controlled release profile in certain circumstances.

The cubic phase structure within cubosomes provides a lipid bilayer motif repeatedly wrapped to a triply periodic minimal surface. The increased surface curvature of the lipid membrane within these particles of the first aspect may assist in promoting bilayer fusion upon contact with other self-assembled systems, including lipid membranes such as bacterial membranes. High curvatures values are therefore preferred in the lipid carriers of the present disclosure. Owing to their high internal surface area and amphiphilic nature, cubosomes have the capacity to encapsulate and release a wide array of currently available active agents including antimicrobials such as small molecules, proteins, antimicrobial peptides and other biocidal components.

In terms of the fusogenic lipids used in formation of the particle of the first aspect, the critical packing parameter (CPP) of the lipid(s) can be used to rationalise the mean and Gaussian curvatures, being a property of the formed particle, and so indicate the nature of the mesophase formed or being formed and allows for considerations of suitability of the resulting non-lamellar LLC particle as an active agent carrier. The CPP is related to the mean and Gaussian curvatures via the following equation:

$$C P P = 1 + H {l}_{c} + \frac{K {l}_{c}^{2}}{3}$$

where:
lc is the effective length of the hydrocarbon chain
H is the Mean curvature
K is the Gaussian curvature

The molecular geometry of the relevant amphiphile lipid for use in forming the particles of the disclosure should satisfy the above equation to yield CPP>1.

The same approach can be applied for more than one amphiphile lipid. For multiple amphiphiles an amphiphile with an intrinsic CPP less than 1 can be included to a composition with a secondary amphiphile CPP>1, such that the average CPP is >1. Secondary additives which may also contribute to curvature increase to achieve CPP>1 include small hydrophobic molecules, polymers which interact with the amphiphile headgroup, strong kosmotropes and SiRNA an DNA. Conversely, the curvature can be decreased through inclusion of amphiphiles with CPP<1, high molecular weight PEG, strong chaotropes, charged headgroups, and solvents with a Log P between −1.5 and 0.

The LLC particles may thereby be classified based upon their interfacial curvature which may be calculated by approaches known in the art. In general terms, the curvature of the inverse lyotropic phases increases in the order lamellar<bicontinuous cubic<hexagonal<micellar cubic.

In embodiments, the one or more fusogenic amphiphilic lipids have a critical packing parameter (CPP) about or greater than 1.0.

In embodiments, the one or more fusogenic amphiphilic lipids have a CPP of between about 1.0 to about 3.0, preferably between about 1.0 to about 2.5, more preferably between about 1.0 to about 2.0, even more preferably between about 1.0 to about 1.75, still yet more preferably between about 1.0 to about 1.5.

It will be appreciated that when multiple lipids are incorporated into a particle of the first aspect then all references above to CPP values of individual lipids become a reference to the average CPP value. That is, when the LLC particle comprises more than one amphiphiles (lipid), an average CPP may be defined as the molar average of all the CPP values of the constituent amphiphile lipids. The average CPP values may be selected from those provided above.

In embodiments, the non-lamellar lyotropic liquid crystalline phase particle has an average CPP value between about 1.0 to about 3.0, preferably between about 1.0 to about 2.5, more preferably between about 1.0 to about 2.0, even more preferably between about 1.0 to about 1.75, still yet more preferably between about 1.0 to about 1.5.

The CPP is calculated as follows: v/a₀l_{c}; where l_{c} is the effective length of the amphiphile (lipid) chain; a₀ is the effective surfactant head group area (determined by the balance of inter-chain attractive and head group repulsive interactions); and v is the average volume occupied by the amphiphile molecule.

Without wishing to be bound by theory, the inventors postulate that an average CPP value between about 1.0 to about 3.0, optimally between about 1.0 to about 1.5, provides for particle curvature which allows for both capture of the active agent and for subsequent release. The CPP value may also allow for a prediction of the likelihood of the particle fusing with a biological membrane.

Similarly, the spontaneous splay value correspondences to the spontaneous curvature of the non-lamellar LLC particle. When two lipids have corresponding spontaneous splay energies then fusion between them becomes more energetically favourable. It is therefore believed that particles of the first aspect having the following splay values will be more likely to undergo a desired fusion event with a biological membrane, such as a bacterial membrane.

The choice of fusogenic lipid will clearly affect splay and can be determined based on, for example, the selection of hydrophobes to enhance chain splay including employing unsaturated hydrophobes such as myristyl, pentadecenyl, oleyl, elaidyl, linoleyl, linolenyl, arachindonyl, docosenyl and/or isoprenoid-type hydrophobes such as 3,7,11-trimethyl-dodecyl, 5,9,13-trimethyltetradecanyl, 3,7,11,15-tetramethyl-hexadecyl, 5,9,13,17-tetramethyloctadecyl. Non-limiting examples of such lipids include ME, MP, MM, MV, MO, ML and MR, as are known in the art.

The energy cost of per surface area due to splay can be approximated by

fₛ=½·κ·(div n [symbol] )²

Where κ is the splay modulus of the monolayer, [symbol] is the spontaneous splay.

fₛ=½·κₜ·t²

Kₜ is the splay modulus of the monolayer, t is the tilt vector.

### The Total Energy Cost

fₜₒₜ=½·κ·(div n [symbol] )²+½·κₜ·t²−½·κ˜ [symbol] ²

In embodiments, the non-lamellar lyotropic liquid crystalline phase particle has an internal curvature induced splay ( [symbol] ) less than −0.05 nm⁻¹.

In embodiments, the non-lamellar lyotropic liquid crystalline phase particle has an internal curvature induced splay ( [symbol] ) less than −0.10 nm⁻¹.

In embodiments, the non-lamellar lyotropic liquid crystalline phase particle has an internal curvature induced splay ( [symbol] ) less than −0.15 nm⁻¹.

In embodiments, the non-lamellar lyotropic liquid crystalline phase particle has an internal curvature induced splay ( [symbol] ) less than −0.20 nm⁻¹.

In embodiments, the non-lamellar lyotropic liquid crystalline phase particle has an internal curvature induced splay ( [symbol] ) less than −0.25 nm⁻¹.

In embodiments, the non-lamellar lyotropic liquid crystalline phase particle has an internal curvature induced splay ( [symbol] ) between about −0.05 nm⁻¹ to about −0.95 nm⁻¹, or between about −0.05 nm⁻¹ to about −0.85 nm⁻¹, or between about −0.05 nm⁻¹ to about −0.75 nm⁻¹, or between about −0.05 nm⁻¹ to about −0.65 nm⁻¹, or between about −0.05 nm⁻¹ to about −0.55 nm⁻¹, or between about −0.05 nm⁻¹ to about −0.40 nm⁻¹, or between about −0.10 nm⁻¹ to about −0.95 nm⁻¹, or between about −0.10 nm⁻¹ to about −0.85 nm⁻¹, or between about −0.10 nm⁻¹ to about −0.75 nm⁻¹, or between about −0.10 nm⁻¹ to about −0.65 nm⁻¹, or between about −0.10 nm⁻¹ to about −0.55 nm⁻¹, or between about −0.10 nm⁻¹ to about −0.40 nm⁻¹, or between about −0.15 nm⁻¹ to about −0.75 nm⁻¹, or between about −0.15 nm⁻¹ to about −0.65 nm⁻¹, or between about −0.15 nm⁻¹ to about −0.55 nm⁻¹, or between about −0.15 nm⁻¹ to about −0.40 nm⁻¹, or between about −0.20 nm⁻¹ to about −0.75 nm⁻¹, or between about −0.20 nm⁻¹ to about −0.65 nm⁻¹, or between about −0.20 nm⁻¹ to about −0.55 nm⁻¹, or between about −0.25 nm⁻¹ to about −0.75 nm⁻¹, or between about −0.25 nm⁻¹ to about −0.65 nm⁻¹, or between about −0.25 nm⁻¹ to about −0.55 nm⁻¹.

Further, a person of skill in the art will, in light of the present disclosure, be able to use the following equation to ascertain appropriate curvature levels to provide the benefits described herein:

c_{0T}=xc₀ᵢ+(1−x)c₀ⱼ

where c_{0T} is total curvature, x is fractional composition lipid i, c₀ᵢ is curvature of lipid i, c₀ⱼ, is curvature of lipid j, (1−x) is fractional composition of lipid j. For further combinations the equation is expanded to include lipid k; where the sum of compositions equals 1.

In embodiments, the lattice parameter of the non-lamellar lyotropic liquid crystalline phase particle is between about 20 to about 684 Å, or between about 20 to about 500 Å, or between about 20 to about 400 Å, or between about 20 to about 200 Å, or between about 20 to about 190 Å, or between about 20 to about 180 Å, or between about 20 to about 170 Å, or between about 20 to about 160 Å, or between about 20 to about 150 Å, or between about 40 to about 684 Å, or between about 40 to about 500 Å, or between about 40 to about 400 Å, or between about 40 to about 200 Å, or between about 40 to about 190 Å, or between about 40 to about 180 Å, or between about 40 to about 170 Å, or between about 40 to about 160 Å, or between about 40 to about 150 Å, or between about 60 to about 684 Å, or between about 60 to about 500 Å, or between about 60 to about 400 Å, or between about 60 to about 200 Å, or between about 60 to about 190 Å, or between about 60 to about 180 Å, or between about 60 to about 170 Å, or between about 60 to about 160 Å, or between about 60 to about 150 Å, or between about 80 to about 684 Å, or between about 80 to about 500 Å, or between about 80 to about 400 Å, or between about 80 to about 200 Å, or between about 80 to about 190 Å, or between about 80 to about 180 Å, or between about 80 to about 170 Å, or between about 80 to about 160 Å, or between about 80 to about 150 Å, or between about 100 to about 684 Å, or between about 100 to about 500 Å, or between about 100 to about 400 Å, or between about 100 to about 200 Å, or between about 100 to about 190 Å, or between about 100 to about 180 Å, or between about 100 to about 170 Å, or between about 100 to about 160 Å, or between about 100 to about 150 Å, or between about 120 to about 684 Å, or between about 120 to about 500 Å, or between about 120 to about 400 Å, or between about 120 to about 200 Å, or between about 120 to about 190 Å, or between about 120 to about 180 Å, or between about 120 to about 170 Å, or between about 120 to about 160 Å, or between about 120 to about 150 Å.

Lattice parameters can, for example in relation to sponge particles which typically have larger lattice parameters than cubosomes or hexosomes, be swollen in ranges up to 684 Å. More commonly swollen phases may have values from 200-400 Å. Swollen lattice parameters have certain design rules including that the head group could contain electrostatic charges and these can be negatively charged (e.g. PG and PS phospholipids) or positively charged (e. g. DOTAP and DOMA). The head group may comprise hydration agents with multiple hydroxyl groups (e.g. DGMO and OG). The hydrophobic region may comprise cholesterol or other stiffening agents to stabilise the membrane and/or may comprise amphiphiles that promote a decrease in membrane curvature (e.g. PC and PE phospholipids). Lipid-PEG polymers (e.g. DOPE-PEG and MO-PEG) could be used in combination with charged lipids to swell the water channels. Further, block copolymers (e.g. Pluronic F127, F108 and Polysorbate 80) could be used as stabilisers when nanoparticle dispersions are required, although they may not have a direct effect on swelling the water channels

In embodiments, at least one of the one or more lipids presents, or has been modified to present, a charge.

In embodiments, the non-lamellar lyotropic liquid crystalline phase particle may further comprise a non-amphiphilic and/or non-fusogenic charged compound.

The presence of a charged species has been found to have beneficial results in terms of promoting contact between particles of the first aspect and biological membranes, as shown in the results. This is believed to be due to a positive charge on the non-lamellar LLC particle assisting in overcoming any steric or electrostatic barrier which might otherwise keep the particle and biological membrane apart. The number of contact events can thereby be increased resulting in a correspondingly greater number of fusion and delivery events. This is particularly important with bacterial membranes, such as gram-negative bacterial membranes, which present a net negative charge.

The charge may be generated by incorporation, into the non-lamellar LLC particle, of one or more charged species. This may be achieved by inclusion of a charged amphiphile or surfactant. It will be appreciated that a wide range of cationic lipids, surfactants and related compounds which are known in the art, may be appropriate.

In embodiments, the non-lamellar LLC particle may include a cationic lipid.

In embodiments, the cationic lipid may be a lipid comprising a nitrogen-containing head group capable of bearing a positive charge.

A positively charged species, such as the head group of an amphiphilic or surfactant molecule, is also postulated to be of importance in generating an initial perturbation of the biological membrane with which it is hoped to fuse the non-lamellar LLC particle of the first aspect. This may involve a bending, tilting, splaying or the like of the membrane which can then be an initiation event to fusion.

In embodiments, the cationic lipid may be incorporated into the non-lamellar LLC particle at an amount between 0.1 to less than 20 mol %, or between 0.1 to less than 10 mol %, or between 0.1 to less than 5 mol %, or between 0.1 to less than 4 mol %, or between 0.1 to less than 3 mol %, or between 0.1 to less than 2 mol %, or between 0.5 to less than 20 mol %, or between 0.5 to less than 10 mol %, or between 0.5 to less than 5 mol %, or between 0.5 to less than 4 mol %, or between 0.5 to less than 3 mol %, or between 0.5 to less than 2 mol %.

In embodiments, the lipid carrier which may be a non-lamellar lyotropic liquid crystalline phase particle may have a zeta potential which is greater than 0 mV.

In embodiments, the non-lamellar lyotropic liquid crystalline phase particle may have a zeta potential of greater than about 0 mV to about 60 mV, greater than about 0 mV to about 50 mV, greater than about 0 mV to about 40 mV, between about 1 mV to about 60 mV, between about 1 mV to about 50 mV, between about 1 mV to about 40 mV.

The inventors further postulate that, to achieve appropriate fusion of the particle of the first aspect with a biological membrane such as the membrane of a gram-negative bacterium, a mycobacterium or a fungi, design and control of the viscosity of the particle of the first aspect is an important parameter to consider. Particularly, once the particles of the first aspect are in contact with the biological membrane it is important to ensure they have sufficient viscosity to achieve sufficient adhesion and ensure they do not part. This will also ensure that the nanostructure is maintained under most flow conditions.

In embodiments, the non-lamellar lyotropic liquid crystalline phase particle may have a dynamic viscosity of between about 10 Pa s⁻¹ to about 1×10\hat{ }Pa s⁻¹ at between 0° C. to 40° C.

In embodiments, the non-lamellar lyotropic liquid crystalline phase particle may have a shear viscosity of between about 5 cSt to about 30,000 cSt, or between about 10 cSt to about 20,000 cSt; at shear rates less than 100 s⁻¹.

The one or more fusogenic amphiphilic lipids may be selected from those which are known in the art to form, particularly, cubosomes and hexosomes. The selection of the appropriate one or more fusogenic amphiphilic lipids may be made on the basis of certain requirements which are understood in the art. For example, the lipid(s) may be chosen from those which adopt a Type II lyotropic liquid crystalline phase at ambient and physiological temperatures. Parameters which may be appropriate for selection of an appropriate lipid include (i) on the hydrophobic component: 1. The temperature should be above the chain melting temperature such that molten chains are present; and 2. There should be at least one cis unsaturated bond in a carbon chain of at least 14 carbons at a position at least mid-way along the backbone; or 3. The carbon backbone should contain at least 12 carbons of which 3 are secondary carbons with methyl branches; and 4. The molecular weight of the hydrophobe should be at least >200 amu; and (ii) in relation to the head group: 5. The head group should contain at least three functional groups with minimum hydrophilicity (e.g. hydroxyl); 6. The head group should be able to form head group-water hydrogen bond networks; and 7. The head group area should be small relative to the hydrophobe footprint. By way of a guide, this is exemplified by the MO lipid used in the examples of the present disclosure as it: fulfils criteria 1, 2 and 4 for the hydrophobe; and fulfils criteria 5, 6 and 7 for the head group. It will be appreciated that many other lipids are available which fulfil these criteria appropriately and they may be selected on the basis of these criteria which are known, or easily ascertained, values.

Guidance may be found in one or more of the following publications which are each incorporated by reference herein in their entirety: (i) T. Kaasgaard and C. J. Drummond “Ordered 2D and 3D Nanostructured Amphiphile Self-Assembly Materials Stable in Excess Solvent” Phys. Chem. Chem. Phys. 2006, 8, 4957-4975. (ii) C. Fong, T. Le and C. J. Drummond “Lyotropic Liquid Crystal Engineering—Ordered Nanostructured Small Molecule Amphiphile Self-Assembly Materials by Design” Chem. Soc. Rev., 2012, 41, 1297-1322 DOI: 10.1039/c1cs15148g; (iii) L. van ′t Hag, S. L. Gras, C. E. Conn and C. J. Drummond “Lyotropic liquid crystal engineering moving beyond binary compositional space—Ordered nanostructured amphiphile self-assembly materials by design” Chem. Soc. Rev., 2017, 46, 2705-2731. DOI: 10.1039/c6cs00663a; and (iv) S. Sarkar, N. Tran, Md H. Rashid, T. C. Le, I. Yarovsky, C. E. Conn and C. J. Drummond “Toward cell membrane biomimetic lipidic cubic phases: a high-throughput exploration of lipid compositional space” ACS Applied Biomaterials, 2019, 2, 182-195. DOI: 10.1021/acsabm.8b00539.

Poly-hydroxyl (glycolipids) and polyethers (polyethylene oxides) form two of the largest categories of Type II forming head groups. Non-limiting examples of head group motifs include alcohols, fatty acids, monoacylglycerides, MAGs, 2-MAGs, glycerates, glyceryl ethers, ethylene oxides, amides, monoethanolamides, diethanolamides, serinolamides, methylpropanediolamides, ethylpropanediolamides, ureas, urea alcohols, biurets, biuret alcohols, ureides, endocannabinoids (anandamide, virodhamine, 2-glycerol, dopamine, 2-glycerol ether) and glycolipids. Examples include phospholipids such as DMPC and DMPE.

In embodiments, the one or more fusogenic amphiphilic lipids may be selected from the group consisting of ethylene oxide-, monoacylglycerol-, glycolipid-, phosphatidylethanolamine-, and urea-based amphiphiles, and derivatives or analogues thereof.

Ethylene oxide amphiphiles may include C₁₂(EO)₂, C₁₂(EO)₄, C₁₂(EO)₅, and C₁₂(EO)₆ and dialkyl ethylene oxide amphiphiles. Monoacylglycerols may include monomyristolein, monoolein, monovaccenin and monoerucin. Amphiphiles resembling monoacylglycerols may be appropriate and include oleyl glycerate, phytanyl glycerate, glyceryl monooleyl ether, glyceryl phytanyl ether, phytantriol and monononadecenoin. Glycolipids with sugar moieties which may be appropriate including monosubstituted glycolipids: β-Mal₃(Phyt)₂, β-Glc(Phyt), β-Xyl(Phyt), β-Glc-(TMO)₂, β-Mal₂(Phyt)₂ and β-Glc(Phyt)₂; and disubstituted unbranched glycolipids: 1,2-diacyl-(β-D-glucopyranosyl)-sn-glycerols; 1,2-dialkyl-(β-D-glucopyranosyl)-sn-glycerols; 1,3-diacyl-(β-D-glucopyranosyl)-sn-glycerols; 1,3-dialkyl-(β-D-glucopyranosyl)-sn-glycerols. Phosphatidylethanolamine amphiphiles may include dioleoylphosphatidylcholine (DOPC) and dioleoylphosphatidylethanolamine (DOPE). Urea amphiphiles may include dodecylurea (DU), octadecylurea (ODU), oleylurea (OU), oleylbiuret (OBU), linoleylurea (LU), phytanylurea (PU), hexahydrofarnesyl-urea (HFU).

In embodiments, the one or more fusogenic amphiphilic lipids may be selected from the group consisting of 1-monoolein, 2-monoolein, citrem, oleoyl lactate, oleamide, monoelaidin, linoleic acid, elaidic acid, monopalmitolein, monolinolein, phytantriol, diolein, triolein, dioleoyl-glycerol, didodecyldimethylammonium bromide, dioctadecyl (dimethyl) ammonium chloride (DOAC/DODMAC) or bromide (DODAB), 1,2-dioleoyl-sn-glycero-3-phosphoethanolamine (DOPE), 1,2-Dioleoyl-phosphatidylglycerol (DOPG), oleic acid, lysol-hydroxy-2-oleoyl-sn-glycero-3-phosphocholine, 1,2-dioleoyl-sn-glycero-3-dihexyl-phosphocholine, vitamin E tocopherol, vitamin E (tocopheryl) acetate, phytanoyl monoethanolamide, farnesoyl monoethanolamide, oleoyl monoethanolamide, linoleoyl monoethanolamide and linolenoyl monoethanolamide.

Single-chain amphiphile lipids may be selected from the group consisting of saturated fatty acids C7-C16, oleic acid, elaidic acid, linoleic acid, sodium/gadolinium oleate, oleamide, 1-glyceryl monooleyl ether (GME), GMO, 2-MO, oleoyl lactate, citrem, Diglycerol monooleate (DGMO), Lyso (1-oleoyl)-phosphatidyl-choline, (Z)-Octadec-9-enylferrocene, N-Dodecyl-caprolactam (C12), Vitamin K1, ubiquinone-10 (coenzyme Q10), Vitamin E, Vitamin E acetate, Vitamin A palmitate, Alpha-tocopheryl PEO1000 succinate (vitamin E TPGS), PEG2000-MO, PEG-PT, PEOₓ-stearate (x=40-100), polysorbate 80.

Amphiphile lipids with multiple alkyl chains may be selected from the group consisting of didodecyldimethylammonium bromide (DDAB); Di(canola ethyl ester) dimethyl ammonium chloride (DEEDAC); Dioctadecyl (dimethyl) ammonium chloride (DOAC/DODMAC) or bromide (DODAB); diolein; Dioleoyl-glycerol (DOG), EDTA-bi-oleoyl; EDTA-bi-phytanyl; 1,2-Dioleoyl-3-trimethylammonium-propane (DOTAP); 1,2-Dioleoyl-phosphatidic acid (DOPA); 1,2-Dioleoyl-phosphatidylglycerol (DOPG), 1,2-Distearoyl-phophatidylglycerol (DSPG); 1,2-Dioleoyl-phosphatidylethanolamine (DOPE), 1,2-distearoyl-glycero-3-phosphoethanolamine (DSPE); 1,2-Dioleoyl-phosphatidylcholine (DOPC); 1-Palmitoyl-2-oleoyl-sn-glycero-3-phosphocholine (POPC); 1,2-Dioleoyl-sn-glycero-3-phosphoserine (DOPS); 1,2-Dipalmitoylphosphatidylserine (DPPS); DSPE-mPEG350, 750, 2000 (X=7,16 or 45); DSPE-PEG2000, 3400, 5000; DMPE-mPEG550; (C18)2 DTPA (Gd), Cardiolipin, Cyclodextrin derivative (βCD-nC₁₀).

In any embodiment herein, the fusogenic amphiphilic lipid may be a monoolein and/or phytantriol.

In any embodiment herein, the lipid particles of the present disclosure may comprise MO or phytantriol in combination with one or more of cholesterol, DLPC, DSPC, DPPE, DPPS, DOPS, DPPC, DMPC, DMPS and DLPS.

Monoacylglycerols are known to form reversed phases over large regions of their phase diagrams, with monoolein being the most prominent. Formation of reversed phases is favoured because of the kink that is introduced by the cis-double bond. The longer acyl chain increases the hydrophobic chain volume and makes monoolein more wedge-shaped and shifted towards type 2 phases in the spectrum of mesophases. If the double bond is closer to the end of the lipid it diminishes its effect and makes it less wedge-shaped. Acyl chain extension is expected to drive the mesophase formation further towards the type 2 phases, and on this basis it is not surprising that the H2-phase becomes the dominant phase with such a change.

In embodiments, wherein the non-lamellar lyotropic liquid crystalline phase particle comprises at least two fusogenic amphiphilic lipids, then at least one of the fusogenic amphiphilic lipids may be selected from a monoolein and phytantriol.

In such embodiments, the monoolein and/or phytantriol fusogenic amphiphilic lipids may, individually or in combination, be combined with one or more of triolein, vitamin E and DOPE. If it is desired to present a charge on the non-lamellar lyotropic liquid crystalline phase particle then these combinations may, themselves, further be combined with one or more cationic lipids selected from those which are well-known in the art and commercially available and including a wide variety of quaternary ammonium cationic compounds.

Certain of the lipids may be chosen particularly because of their effect on the internal curvature of the final lipid particle, for example DOPE. When such lipids are included in addition to the main fusogenic ipid, such as monoolein, then they may be present at approximately 10 to 40 mol %.

Representative cationic lipids may be selected from the following non-limiting examples: 3-β[⁴N (¹N⁸-diguanidinospermidine)-carbamoyl] cholesterol (BGSC); 3-β [N,N-diguanidinoethyl-aminoethane)-carbamoyl] cholesterol (BGTC); N,N¹,N², N³ tetra-methyltetrapalmitylspermine (cellfectin); NtN′-butyl-N′-tetradecyl-3-tetradecyl-aminopropion-amidine (CLONfectin) Dimethyldioctadecyl ammonium bromide (DDAB); 1,2-myristoxypropyl-3-dimethyl-hydroxyethylammonium bromide (DMRIE); 2,3-dioleoyloxy-N-[2 (sperminecarboxamide) ethyl]-N,N-dimethyl-1-p-ropana (Nitrotrifluoroacetate) (DOSPA); 1,3-dioleoyloxy-2-(6-carboxyspermyl)-propylamide (DOSPER); 4-(2,3-bis-palmitoyloxy-propyl)-1-Methyl-1H-imidazole (DPIM); N,N,N′,N′-tetramethyl-N,N′-bis (2-hydroxyethyl)-2,3-dioleoyloxy-1,4-butane Diammonium iodide (Tfx-50); N-1-(2,3-dioleoyloxy) propyl-N,N, N-trimethylammonium chloride (DOTMA) or other N—(N,N-1-Dialkoxy)-alkyl-N,N, N-trisubstituted ammonium surfactants; trimethylammonium groups are double-stranded (DOT) via a butanol spacer arm 1,2-dioleoyl-3-(4′-trimethylammonio) butanol-sn-glycerol (DOBT) or cholesteryl (4′-trimethylammonia) butanoate (ChOTB) connected to a cholesteryl group (for ChOTB) DORI (DL-1,2-dioleoyl-3-dimethylaminopropyl-β-hydroxyethylammonium) or DORIE (DL-1,2-O-dioleoyl-3-dimethylaminopropyl) as disclosed in WO 93/03709-B-hydroxyethylammonium) (DORIE) or analogs thereof; 1,2-dioleoyl-3-succinyl-sn-glycerol choline ester (DOSC); tetraoctylammonium bromide (TOAB) as a cationic phase transfer agent; cholesteryl hemisuccinate ester (ChOSC); Midonglycylspermine (DOGS) and dipalmitoylphosphatidylethanol amylspermine (DPPES) or cationic lipids disclosed in U.S. Pat. No. 5,283,185, cholesteryl-3β-carboxy-amido-ethylenetrimethylammonium chloride, 1-dimethyl Amino-3-trimethylammonio-DL-2-propyl-cholesterylcarboxylate iodide, cholesteryl-3-O-carboxyamidoethyleneamine, cholesteryl-3-β-oxysuccinamide-ethylenetrimethylammonium iodide, 1-Dimethylamino-3-trimethylammonio-DL-2-propyl-cholesteryl-3-β-oxysuccinate iodide, 2-(2-trimethylammonio)-ethylmethylaminoethyl-choles Teryl-3-β-oxysuccinate iodide, 3-β-N— (N′,N′-dimethylaminoethane) carbamoylcholesterol (DC-chol), and 3-β-N-(polyethyleneimine)-carbamoyl Cholesterol; O, O-Dimyristyl-N-lysyl-aspartate (DMKE); O, O-Dimyristyl-N-lysyl-glutamate (DMKD): 1,2-Dimyristyloxypropyl-3-dimethyl-hydroxyethylammonium bromide (DMRIE); 1,2-dilauroyl-sn-glycero-3-ethylphosphocholine (DLEPC); 1,2-dimyristyl-sn-glycero-3-ethylphosphocholine (DMEPC); 1,2-dioleoyl-sn-Glycero-3-ethylphosphocholine (DOEPC); 1,2-dipal Toyl-sn-glycero-3-ethylphosphocholine (DPEPC); 1,2-distearoyl-sn-glycero-3-ethylphosphocholine (DSEPC); 1,2-dioleoyl-3-trimethylammoniumpropane (DOTAP); Dioleoyl dimethylaminopropane (DODAP); 1,2-palmitoyl-3-trimethylammoniumpropane (DPTAP); 1,2-distearoyl-3-trimethylammoniumpropane (DSTAP); 1,2-myristoyl-3-trimethyl Ammonium propane (DMTAP); and sodium dodecyl sulfate (SDS).

In embodiments, particularly preferred cationic lipids are DOTAP and/or DODAB and/or tetraoctylammonium bromide (TOAB).

In embodiments the non-lamellar lyotropic liquid crystalline phase particle may comprise a fatty acid in addition to one or more fusogenic lipids such as, for example, oleic acid.

In embodiments, the non-lamellar lyotropic liquid crystalline phase particle may comprise at least one stabiliser. The stabiliser may be selected from those known in the art.

Preferably, the stabiliser is a Poloxamer or a surfactant or a PEGylated lipid stabilizer, or a modified version of these.

In embodiments, the stabiliser is selected from a PEG-PPO-PEG triblock copolymer and a non-ionic block copolymer surfactant and a PEO co-polymerised with a charged moiety. Poloxamer 407 and Pluronic 127 may be suitable examples of a stabilising agent and may be incorporated into any of the embodiments of the first aspect described herein. PEO co-polymerised with (3-Acrylamidopropyl)trimethylammonium chloride, or a similar charge-carrying moiety, may also be appropriate. PEGylated lipid stabilisers are also appropriate including but not limited to PEG2000-MO, PEG-PT, DSPE-PEG(2000) Amine, 18:0 PEG2000 PE, and DSPE-PEG(5000) Amine. Many other such stabilisers are known in the art.

The use of a stabiliser is preferable and while the nature of the stabiliser may be selected based on the nature of the lipids, the selection and understanding of the compatibility of these components can be based upon information known in the art.

The many steric stabilisers which have been reported to date can be divided into four groups: (i) amphiphilic block copolymers (i.e. Poloxamer™), (ii) PEGylated lipids (iii) customized lipid-copolymers and (iv) alternative steric stabilizers (e.g., bile salts, proteins). Ideally the stabiliser selected will prevent aggregation of the particles by providing an electrostatic or, more commonly, steric barrier between approaching particles. Stabilisers which may function optimally in the lipid particles of the present disclosure share similar properties including (i) they are generally highly hydrophilic with a high HLB (hydrophilic-lipophilic balance) value due to an asymmetric amphiphilic polymer structure with a larger hydrophilic domain. It is important that the hydrophilic part of the molecule is not surrounded by hydrophobic regions. A high HLB may be achieved via use of longer PEG chains or multiple PEG chains; (ii) presence of hydrogen bond acceptors and absence of hydrogen bond donors; and (iii) electrically neutral. A person of skill in the art can select the appropriate stabiliser on this basis. Further, the following journal articles address key aspects of stabilisers which may be appropriate for use with the lipid particles of the present disclosure and are hereby incorporated by reference in their entirety: (i) J. Y. T. Chong, X. Mulet, B. J. Boyd and C. J. Drummond; “Steric Stabilizers for Cubic Phase Lyotropic Liquid Crystal Nanodispersions (Cubosomes)” in “Advances in Planar Lipid Bilayers and Liposomes”, Vol 21, Chp 5, (2015) p. 131-187, ISSN 1554-4516, Elsevier; (ii) J. Zhai, B. Fan, S. H. Thang, C. J. Drummond “Novel amphiphilic block copolymers for the formation of stimuli-responsive non-lamellar lipid nanoparticles” Molecules, 2021, 26, 3648-3664. DOI: 10.3390/molecules26123648; (iii) J. Zhai, R. Suryadinata, B. Luan, N. Tran, T. M. Hinton, J. Ratcliffe, X. Hao and C. J. Drummond “Amphiphilic brush polymers produced by the RAFT polymerisation method stabilise and reduce the cell toxicity of lipid lyotropic liquid crystalline nanoparticles” Faraday Discussions, 2016, 191, 545-563. DOI: 10.1039/C6FD00039H; Faraday Discussion 191 on Nanoparticles with Morphological and Functional Anisotropy; (iv) J. Zhai, T. J. Hinton, L. J. Waddington, C. Fong, N. Tran, X. Mulet, C. J Drummond and B. W. Muir “Lipid-PEG Conjugates Sterically Stabilise and Reduce the Toxicity of Phytantriol-Based Lyotropic Liquid Crystalline Nanoparticles” Langmuir, 2015, 31, 10871-10880. DOI: 10.1021/acs.langmuir.5b02797; (v) J. Y. T. Chong, X. Mulet, D. Keddie, L. J. Waddington, S. T. Mudie, B. J. Boyd and C. J. Drummond “Novel Steric Stabilisers for Lyotropic Liquid Crystalline Nanoparticles: Pegylated Phytanyl Copolymers” Langmuir, 2015, 31, 2615-2629. DOI: 10.1021/la501471z; (vi) J. Y. T. Chong, X. Mulet, A. Postma, D. J. Keddie, L. J. Waddington, B. J. Boyd and C. J. Drummond “Novel RAFT Amphiphile Brush Copolymer Steric Stabilisers for Cubosomes: Poly(octadecyl acrylate)-block-poly(polyethylene glycol methyl ether acrylate)” Soft Matter, 2014, 10, 6666-6676. DOI: 10.1039/C4SM01064G; (vii) A. Tilley, C. J. Drummond and B. J. Boyd “Disposition and Association of the Steric Stabiliser Pluronic F127 in Lyotropic Liquid Crystalline Nanostructured Particle Dispersions” J. Colloid and Interface Science, 2013, 392, 288-296. DOI: 10.1016/j.jcis.2012.09.051 (viii) J. Y. T. Chong, X. Mulet, L. J. Waddington, B. J. Boyd and C. J. Drummond “High Throughput Discovery of Novel Steric Stabilisers for Cubic Lyotropic Liquid Crystal Nanoparticle Dispersions” Langmuir, 2012, 28, 9223-9232. DOI: 10.1021/la301874v; (ix) J. Y. T. Chong, X. Mulet, L. J. Waddington, B. J. Boyd and C. J. Drummond “Steric Stabilisation of Cubic Lyotropic Liquid Crystalline Nanoparticles: High Throughput Evaluation of Triblock Polyethylene Oxide-Polypropylene Oxide-Polyethylene Oxide Copolymers.” Soft Matter, 2011, 7, 4768-4777. DOI: 10.1039/c1sm05181d.

The stabiliser may be present during formation of the particle of the first aspect.

The stabiliser may be present at between 5 to 20 wt %, 6 to 18 wt %, 7 to 16 wt %, or 8 to 14 wt %.

In embodiments, the lipids forming the particle of the first aspect may substantially comprise the following:
(a) Monolein and/or phytantriol;
(b) Monolein and/or phytantriol, and DOPE;
(c) Monolein and/or phytantriol, and DOTAP;
(d) Monolein and/or phytantriol, and TOAB;
(e) Monolein and/or phytantriol, and oleic acid;
(e) Monolein and/or phytantriol, and DOPE and DOTAP.

In embodiments wherein the lipid particle of the first aspect comprises DOPE then it may be present at between 10 to 40 mol %.

In embodiments wherein the lipid particle of the first aspect comprises DOTAP then it may be present at between 0.5 to 5 mol %, or 0.5 to 4 mol %.

In certain embodiments, the lipids forming the particle of the first aspect may substantially comprise the following:
a) Monoolein; or
b) Monoolein (80-99.9 mol %), triolein (0.1-20 mol %); or
c) Monoolein (80-99.9 mol %), vitamin E (0.1-20 mol %); or
d) Monoolein (80-99.9 mol %), DOPE (0.1-20 mol %).

In certain embodiments, the lipids forming the particle of the first aspect may substantially comprise the following:
a) Monoolein (95-99.9 mol %), DOTAP (0.1-5 mol %); or
b) Monoolein (95-99.9 mol %), DODAB (0.1-5 mol %); or
c) Any lipid from the above lists with DOTAP (0.1-5 mol %).

The non-lamellar lyotropic liquid crystalline phase particle may consist or consist essentially of monoolein and/or phytantriol and one or two amphiphilic lipids selected from the list of fusogenic amphiphilic lipids above.

In embodiments, the one or more fusogenic amphiphilic lipids may be selected from those presenting a hydrophobic tail group selected from the group consisting of oleoyl, linoleoyl, linolenoyl, phytanoyl, farnesoyl or extended aliphatic hydrophobic chain.

In such embodiments, the head group of the fusogenic amphiphilic lipid may be a ‘non-traditional’ head group modified to present a charge or other desirable surface functionality of chemical moiety.

In embodiments wherein at least one fusogenic amphiphilic lipid presents a non-traditional head group then it may be a peptide headgroup or a cationic headgroup not typically associated with the attached hydrophobic tail. In one non-limiting example, the head group may be an aminoglycoside-based head group.

In embodiments, the hydrophobic tail of the fusogenic amphiphilic lipid may be a ‘non-traditional’ tail group modified to present a particular surface functionality or physical characteristic. In particular, the tail group may be modified to allow for control over the final CPP value for the particle. Therefore, in embodiments, the hydrophobic tail of the fusogenic amphiphilic lipid may be selected such that a CPP value of between about 1.0 to about 3.0, preferably between about 1.0 to about 2.5, more preferably between about 1.0 to about 2.0, even more preferably between about 1.0 to about 1.75, still yet more preferably between about 1.0 to about 1.5, is provided for.

In embodiments wherein the non-lamellar lyotropic liquid crystalline phase particle comprises one or more charged amphiphiles or surfactants, fusogenic or otherwise, they may be selected from the group consisting of DOTAP, DOTMA, DODAP, Dioctadecyl (dimethyl) ammonium chloride (DOAC/DODMAC) or bromide (DODAB), 1,2-dioleoyl-sn-glycero-3-phosphoethanolamine (DOPE) and CTAB.

In embodiments, the active agent may be selected from any substance which can affect or assist in identifying any physical or biochemical properties of a biological system, pathway, molecule, or interaction relating to an organism, including but not limited to animals and humans. In particular, as used herein, agents include but are not limited to any substance intended for diagnosis, cure, detection, mitigation, treatment, or prevention of disease in humans or other animals, or to otherwise enhance physical or mental well-being of humans or animals. Examples of biologically active molecules include, but are not limited to, peptides, proteins, dyes, enzymes and small molecule drugs. Classes of active agents that are suitable for use with the methods and compositions described herein include, but are not limited to, drugs, prodrugs, radionuclides, imaging agents, polymers, antibiotics, fungicides, metal-containing nanoparticles, anti-inflammatory agents, anti-tumor agents, cardiovascular agents, anti-anxiety agents, hormones, growth factors, steroidal agents, gene expression modifiers, knockdown agents, siRNA, RNAi agents, dicer substrates, miRNA, shRNA, antisense oligonucleotides, aptamers, microbially derived toxins, antibodies and fragments thereof including anti-tuberculosis antibody fragments, and the like.

Particularly preferred active agents may include any agents known to be effective against gram-negative bacteria and/or mycobacteria and/or fungi.

The active agent may be present at between 0.1 to 30.0 mol %, or between 0.1 to 25.0 mol %, or between 0.1 to 20.0 mol %, or between 0.1 to 10.0 mol %, or between 0.1 to 5.0 mol %, or between 0.1 to 4.0 mol %, or between 0.1 to 3.5 mol %, or between 0.1 to 3.0 mol %, or between 0.5 to 30.0 mol %, or between 0.5 to 25.0 mol %, or between 0.5 to 20.0 mol %, or between 0.5 to 10.0 mol %, or between 0.5 to 5.0 mol %, or between 0.5 to 4.0 mol %, or between 0.5 to 3.5 mol %, or between 0.5 to 3.0 mol %.

It is one advantage of the present invention that active agents may be employed which would otherwise not be able to be used as the “free” active. For example, certain antibiotics are known to bind to serum proteins to such an extent that their efficacy is significantly reduced. Alternatively, some active agents may be active against a target when within a bacterial cell but they are not able to cross the bacterial membrane. The present approach provides for a lipid carrier particle which both protects the active agent from degradation or becoming undesirably bound to serum proteins and also provides for improved delivery into the bacterial or fungal cell.

It will be appreciated that the term “agent” or “active agent”, may include an active compound in the form of a pharmaceutically effective or acceptable salt.

In any embodiment herein, the active agent is not a metal nanocrystal.

Therefore, in one embodiment, there is provided a non-lamellar lyotropic liquid crystalline phase particle comprising one or more fusogenic amphiphilic lipids, and the particle encapsulating an active agent, wherein the particle comprises: (i) an average CPP of between about 1.0 to about 3.0; (ii) a zeta potential which is greater than 0 mV; (iii) and a dynamic viscosity of between about 10 Pa s⁻¹ to about 1 e6 Pa s⁻¹ at between 0° C. to 40° C.

In such embodiments, the average CPP value may be between about 1.0 to about 2.5, more preferably between about 1.0 to about 2.0, even more preferably between about 1.0 to about 1.75, still yet more preferably between about 1.0 to about 1.5.

In embodiments, there is provided a non-lamellar lyotropic liquid crystalline phase particle comprising one or more fusogenic amphiphilic lipids, and the particle encapsulating an active agent, wherein the particle has: (i) an internal curvature induced splay ( [symbol] ) between about −0.10 nm⁻¹ to about −0.55 nm-1; (ii) a zeta potential which is greater than +0 mV; (iii) and a lattice parameter between about 60 to about 684 Å.

In embodiments, the non-lamellar lyotropic liquid crystalline phase particle has a particle diameter of between 50 nm to 450 nm, 80 nm to 400 nm, 100 nm to 300 nm or 120 nm to 300 nm.

Preferably, the non-lamellar lyotropic liquid crystalline phase particle is a cubosome particle.

Preferably, the active agent is one or more of a gram-negative bacteria active agent or mycobacterial antibacterial active agent or an antifungal agent.

Suitably, the non-lamellar lyotropic liquid crystalline phase particle, which in one embodiment is a cubosome nanocarrier particle, comprises one or more positively charged lipids or stabilisers.

In a second aspect, there is provided a pharmaceutical composition comprising a lipid carrier which may be a non-lamellar lyotropic liquid crystalline phase particle of the first aspect and a pharmaceutically acceptable carrier, diluent and/or excipient.

Suitably, the pharmaceutically acceptable carrier, diluent and/or excipient may be or include one or more of diluents, solvents, pH buffers, binders, fillers, emulsifiers, disintegrants, polymers, lubricants, oils, fats, waxes, coatings, viscosity-modifying agents, glidants and the like.

Diluents may include one or more of microcrystalline cellulose, lactose, mannitol, calcium phosphate, calcium sulfate, kaolin, dry starch, powdered sugar, and the like. Binders may include one or more of povidone, starch, stearic acid, gums, hydroxypropylmethyl cellulose and the like. Disintegrants may include one or more of starch, croscarmellose sodium, crospovidone, sodium starch glycolate and the like. Solvents may include one or more of ethanol, methanol, isopropanol, chloroform, acetone, methylethyl ketone, methylene chloride, water and the like. Lubricants may include one or more of magnesium stearate, zinc stearate, calcium stearate, stearic acid, sodium stearyl fumarate, hydrogenated vegetable oil, glyceryl behenate and the like. A glidant may be one or more of colloidal silicon dioxide, talc or cornstarch and the like. Buffers may include phosphate buffers, borate buffers and carbonate buffers, although without limitation thereto. Fillers may include one or more gels inclusive of gelatin, starch and synthetic polymer gels, although without limitation thereto. Coatings may comprise one or more of film formers, solvents, plasticizers and the like. Suitable film formers may be one or more of hydroxypropyl methyl cellulose, methyl hydroxyethyl cellulose, ethyl cellulose, hydroxypropyl cellulose, povidone, sodium carboxymethyl cellulose, polyethylene glycol, acrylates and the like. Suitable solvents may be one or more of water, ethanol, methanol, isopropanol, chloroform, acetone, methylethyl ketone, methylene chloride and the like. Plasticizers may be one or more of propylene glycol, castor oil, glycerin, polyethylene glycol, polysorbates, and the like.

Reference is made to the Handbook of Excipients 6^{th} Edition, Eds. Rowe, Sheskey & Quinn (Pharmaceutical Press), which provides non-limiting examples of excipients which may be useful according to the invention.

It will be appreciated that the choice of pharmaceutically acceptable carriers, diluents and/or excipients will, at least in part, be dependent upon the mode of administration of the formulation. By way of example only, the composition may be in the form of a tablet, capsule, caplet, powder, an injectable liquid, a suppository, a slow release formulation, an osmotic pump formulation or any other form that is effective and safe for administration.

Preferably, the pharmaceutical composition is a liquid dispersion of the particles of the first aspect. The liquid dispersion may be an aqueous dispersion. The liquid dispersion may be encapsulated within standard capsules known for delivery of liquid formulations.

Suitably, the pharmaceutical composition is for the treatment or prevention of a disease, disorder or condition in a mammal, as described further herein.

In a third aspect, there is provided a method of controlled release of an active agent including the step of forming a non-lamellar lyotropic liquid crystalline phase particle of the first aspect; and administering the non-lamellar lyotropic liquid crystalline phase particle to a target area.

In embodiments, the target area may be a gram-negative bacterial infection, or a mycobacterial infection, or a fungal infection. The infection may be in a mammal.

In a fourth aspect, there is provided a method of forming a non-lamellar lyotropic liquid crystalline phase particle of the first aspect including the steps of (i) providing one or more fusogenic amphiphilic lipids; and (ii) exposing the one or more fusogenic amphiphilic lipids to a solution in the presence of an active agent.

The solution may be an aqueous solution.

In embodiments, the solution may be tailored to be more amenable to the use of hydrophobic active agents. For example, solvent blends may be used.

As discussed, particles of the first aspect may provide for a desirable release profile of an active agent due to the trapping of the active within the complex internal architecture of the non-lamellar LLC particle.

The particles of the first aspect may be formed in a variety of ways depending on exactly what the desired composition is. Briefly, the selected fusogenic lipid(s) may be combined with the appropriate active agent and then exposed to, for example, an aqueous solution to trigger self-assembly. Typically, a stabiliser will also be included in the aqueous solution. Stabilisers such as Poloxamers and Pluronics may be appropriate, including those mentioned above.

If it is desired to include further charged components in the particle then DOTAP or similarly charged species may be included with the fusogenic lipid and active agent.

The nature of these components can be selected to influence the mesophase of the final particle product which will, in turn, influence the release profile of the active agent.

The target area may be any area to which it is desirable to deliver the active agent. Typically, the target area will be within a biological sample or a tissue or fluid of a subject such as a human subject. For example, the target area may be tissue infected with a bacterial infection to which an antibacterial is being delivered via the particles of the first aspect.

It will be appreciated that the final release profile may be best achieved by a number of different particles of the first aspect which have different release profiles of the same active. For example, the parameters discussed above may be selected to provide two different particle populations. One population may be a cubosome population and the other a hexosome population, each carrying the antibacterial. The different populations may be administered separately or together and, due to the different internal architecture and different fusogenic lipids they may deliver the antibacterial at different rates to achieve a therapeutic effect over a longer window of time.

Alternatively, the different populations may comprise different antibacterial agents with the architecture of the particle of the first aspect being tailored to the relevant active.

In a fifth aspect, there is provided a method of treatment or prevention of a disease, disorder or condition including the step of administering a therapeutically effective amount of a non-lamellar lyotropic liquid crystalline phase particle of the first aspect, or the pharmaceutical composition of the second aspect, to a subject in need thereof to thereby treat or prevent the disease, disorder or condition.

In a sixth aspect, there is provided a non-lamellar lyotropic liquid crystalline phase particle of the first aspect, or the pharmaceutical composition of the second aspect, for use in the treatment or prevention of a disease, disorder or condition.

In a seventh aspect, there is provided a use of a non-lamellar lyotropic liquid crystalline phase particle of the first aspect in the manufacture of a medicament for the treatment of a disease, disorder or condition.

As generally used herein, the terms “administering” or “administration”, and the like, describe the introduction of the relevant particle or composition to a mammal such as by a particular route or vehicle. Routes of administration may include topical, parenteral and enteral which include oral, buccal, sub-lingual, nasal, anal, gastrointestinal, subcutaneous, intramuscular and intradermal routes of administration, although without limitation thereto.

By “treat”, “treatment” or treating” is meant administration of the relevant particle or composition to a subject to at least ameliorate, reduce or suppress existing signs or symptoms of the disease, disorder or condition experienced by the subject. to the extent that the medical condition is improved according to clinically acceptable standard(s). For example, “to treat a bacterial infection” means to reduce the infection, or eradicate the infection, or relieve symptoms of the infection in a patient, wherein the improvement and relief are evaluated with a clinically acceptable standardized test and/or an empirical test, including swab sample testing and the like.

By “prevent”, “preventing” or “preventative” is meant prophylactically administering the relevant particle or composition to a subject who does not exhibit signs or symptoms of a disease disorder or condition, but who is expected or anticipated to likely exhibit such signs or symptoms in the absence of prevention. Preventative treatment may at least lessen or partly ameliorate expected symptoms or signs.

As used herein, “effective amount” or “therapeutically effective amount” refers to the administration of an amount of the relevant particle or composition sufficient to prevent the occurrence of symptoms of the condition being treated, or to bring about a halt in the worsening of symptoms or to treat and alleviate or at least reduce the severity of the symptoms. The effective amount will vary in a manner which would be understood by a person of skill in the art with patient age, sex, weight etc. An appropriate dosage or dosage regime can be ascertained through routine trial or based on current treatment regimes for the active being delivered via the particle of the first aspect.

As used herein, the terms “subject” or “individual” or “patient” may refer to any subject, particularly a vertebrate subject, and even more particularly a mammalian subject, for whom therapy is desired. Suitable vertebrate animals include, but are not restricted to, primates, avians, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes). A preferred subject is a human in need of treatment for a disease, disorder or condition as described herein. However, it will be understood that the aforementioned terms do not imply that symptoms are necessarily present. In one embodiment, the subject is a human being treated for a bacterial infection, particularly a gram-negative bacterial infection.

As used herein, the terms “co-therapy” and “combination therapy” shall mean treatment of a subject in need thereof by administering one or more particles or compositions as described herein and one or more agents for treating a disease, disorder or condition by any suitable means, simultaneously, sequentially, separately or in a single pharmaceutical formulation or combination. When administered in separate dosage forms, the number of dosages administered per day for each compound may be the same or different. The relevant particle or composition and one or more active agents for treating a disease, disorder or condition may be administered via the same or different routes of administration.

As discussed above, particles of the first aspect displaying different architectures can be formed and these used to deliver the same or different active agents. This may be particularly appropriate, for example, if treating a multiple-strain bacterial infection where two or more particles may be selected based on their morphology being optimal for delivery of the relevant active and/or ability to fuse with or adhere to the particular strain.

In embodiments, the disease, disorder or condition may be one related to, or caused by a microorganism selected from the group consisting of a gram-negative bacteria, including a mycobacteria, and a fungi.

For the purposes of the present disclosure, mycobacteria are considered to be gram-negative bacteria. While mycobacteria are considered to have cell walls exemplifying aspects of both gram positive and gram-negative bacteria, key mycobacteria such as Mycobacterium tuberculosis may be considered genomically closer to gram-negative bacteria than gram positive: https://doi.org/10.1054/tube.2002.0328 and so this nomenclature is followed herein. Without wishing to be bound by theory it is noted that the main common component of the cell wall is peptidoglycan, which is found in almost all bacteria and is responsible for protective the integrity of the cell. The unique feature of mycobacteria, the complex cell wall, is a well-recognized drug target having unusually strong hydrophobic properties. The crucial core cell wall structure is composed of three main components: arabinogalactan polysaccharide, peptidoglycan and outer layer long-chain, mycolic acids. Approximately 60% of mycobacterial cell membrane is made up of lipids. The mycobacterial cell wall has been shown herein to be amenable to delivery of active agent using the particles of the first aspect.

In one particular embodiment, the disease, disorder or condition is one which is responsive to treatment with antibacterial agents, including antibiotics, and/or antifungals. That is, the disease, disorder or condition is a gram-negative bacterial infection and/or a fungal infection.

In one embodiment, the disease, disorder or condition is caused by, or is associated with, a pathogen. The pathogen may be a gram-negative bacterium or a fungus capable of infecting a mammal.

In embodiments wherein the infection being treated is a gram-negative infection then the causative pathogen may be selected from the group consisting of Enterobacteriaceae, Pseudomonas, Vibrio, Campylobacter, Legionella, Neisseria, Mycobacterium, Hemophilus and Bartonella.

In embodiments, the gram-negative bacteria may be selected from the group consisting of E. coli, Pseudomonas aeruginosa, Klebsiella, Acinetobacter baumannii, Neisseria gonorrhoeae, Enterobacteriaceae, Mycobacterium tuberculosis, and Mycobacterium smegmatis.

Non-limiting examples of fungi include Candida, Cryptococcus and Aspergillus species, although without limitation thereto. In embodiments, the fungus associated with or causing the disease, disorder or condition is selected from the group consisting of C. albicans, C. glabrata, C. parapsilosis, C. tropicalis, C. dublinensis, C. krusei, C. lusitaniae, C. Auris, C. Neoformans and A. fumigatus.

The present inventors have found that the particles of the present disclosure provide advantageous properties which result in fusion events with gram-negative bacteria and fungi but do not follow a similar fusion event with gram-positive bacteria and so, while some active agent may still be delivered to the gram-positive bacteria, they display optimal efficacy against gram-negative and fungal species.

In any aspect or embodiment herein, the active agent may be a hydrophobic active agent.

In embodiments, the active agent is a hydrophobic antibacterial or antifungal active. Preferably, the hydrophobic antibacterial active agent is a gram-negative hydrophobic antibacterial active agent.

Such active agents are well-known in the art and are in common use against gram-negative bacterial and fungal infections. The present disclosure provides for an improved delivery vehicle for such already known antibacterial and antifungal agents and so a person of skill in the art would have no difficulty, based on the present disclosure, in selecting appropriate actives for loading with the particles of the disclosure for subsequent delivery to the site of infection.

When the active agent is an antifungal, the antifungal may be selected from azoles, Echinocandins and amphotericin B. The former two classes are inhibitors to constituents of the fungal cell wall (ergosterol and glucan, respectively) while the latter binds ergosterol.

In embodiments, the active agent is selected from the group consisting of Rifampicin, Fusidic Acid, Ampicillin, Piperacillin, Gentamicin, Vancomycin, Filipin, Amphotericin B, Benzyl Penicillin, Meropenem, Clarithromycin, Novobiocin, Ethambutol, Isoniazid, Streptomycin, Fluconazole, Itraconazole and Pyrazinamide. It will be appreciated that such active agents are representative only.

It will be appreciated that the antibacterial or antifungal agent is released into the gram-negative bacteria or fungi following a fusion event between the non-lamellar lyotropic liquid crystalline phase particle with the outer layer or membrane of said gram-negative bacteria or fungi. That is, the treatment of the gram-negative or fungal infection is not a diffusion release of the active agent in the vicinity of or adjacent to the pathogen. Rather, it is the fusion of the non-lamellar lyotropic liquid crystalline phase particle of the first aspect with the pathogen outer wall, layer or membrane which allows the active agent to be released into the pathogen directly.

In an eighth aspect, there is provided a method of delivering an active agent to a biological target including the step of administering a non-lamellar lyotropic liquid crystalline phase particle of the first aspect.

In one embodiment, the active agent being delivered may be a detection agent and so the method of delivery may be a method of detection.

The detection agent may be a tag, probe or dye.

The particles of the first aspect, active agent and biological target may be as previously described for any of the above aspects. The biological target will, preferably, be a biological sample or an in vivo tissue, organ or fluid from a patient. In embodiments, the patient may have a bacterial infection.

Encapsulation of the active within particles of the first aspect may offer one or more benefits including improving solubility of the active or disguising the solubility issue; protecting the active from destruction or modification in vivo; influence circulation time within a subject; reduce cytotoxicity of the encapsulated active; and reduce the required dosage due to more efficient delivery into the targeted cells.

In embodiments wherein the particles of the first aspect are used to treat a gram-negative bacterial infection then the particle does not enter the bacterium through endocytosis to any significant extent. Put another way, when the particles of the first aspect are used to treat a gram-negative bacterial infection then the active is delivered following, and due to, fusion of the particle with the bacterial membrane.

It will be understood that, in the case of eukaryotic cells, uptake is regulated by a number of endocytosis pathways which envelop and internalise many known nanocarriers or treatment agents. Despite internalisation, cellular processes including transport to lysosomes often leads to decomposition of both the carrier and any encapsulated therapeutic, resulting in low treatment efficiencies. Particles of the first aspect, due to their inherent lipid bilayer motif can, however, subvert these processes by utilising a different internalisation mechanism, namely membrane fusion of the particle's bilayer motif with either external cell walls or within internal endosomes. The direct affinity of particles of the first aspect with cellular membranes can be rationalized by their shared self-assembled nature. While polymer or inorganic nanocarriers are constructed by bonds tethering molecules, non-lamellar LLC particles are objects formed by intermolecular interactions. The former therefore require significantly greater membrane perturbations for internalization. The increased surface curvature of the constituent lipids within non-lamellar LLC particles may specifically promote bilayer fusion. Structurally, yeast cells share a similar inner plasma lipid membrane to gram-negative bacteria being sheathed by an outer layer of chitin, glucan and proteins.

In a ninth aspect of the invention there is provided a method of diagnosing a disease, disorder or condition in a mammal including the step of administering a non-lamellar lyotropic liquid crystalline phase particle of the first aspect or a composition of the second aspect, wherein the active agent within the non-lamellar lyotropic liquid crystalline phase particle of the first aspect is a labelled active agent, to the mammal or to a biological sample obtained from the mammal to facilitate diagnosis of the disease disorder or condition in the mammal.

It will be appreciated from the results shown herein that dyes or labelled agents can be delivered to a range of mammalian cells and so the particles of the first aspect may be useful as detection agents. The particles may also be designed to incorporate molecule tags or targeting moieties such that they are targeted to a certain cell type or receptor.

The following experimental section describes in more detail the characterisation of certain of the compounds of the invention and their efficacy. The intention is to illustrate certain specific embodiments of the compounds of the invention and their efficacy without limiting the invention in any way.

### EXPERIMENTAL

### Reference Sheet

Antibiotics

| | |
|---|---|
| Rif | Rifampicin |
| Gen | gentamicin |
| Novo | Novobiocin |
| pip | Piperacillin |
| Mero | Meropenem |
| DO | Dicloxacillin |
| CLA | Clarithromycin |
| BP | Benzyl penicillin |
| Fil | Filipin |
| AMP | Ampicillin |
| AMPB | Amphotericin B |

Bacteria strains

| | |
|---|---|
| E. coli | Escherichia coli (gram-negative) |
| P. aeruginosa | Pseudomonas aeruginosa (gram-negative, more resistant) |

Fungus

| | |
|---|---|
| C. albicans | candida albicans |

Phase notation

| | |
|---|---|
| Im3m | Primitive cubic phase |
| Pn3m | Diamond cubic phase |
| HII | Inverse hexagonal phase |

Lipids

| | |
|---|---|
| MO | Monoolein (main constituent lipid) |
| PT | Phytantriol (main constituent lipid) |
| DOPE | 1,2-dioleoyl-sn-glycero-3-phosphoethanolamine (high |
| | curvature modifier lipid) |
| DOTAP | Dioleoyl-3-trimethylammonium propane (cationic |
| | lipid modifier) |
| TOAB | tetraoctylammonium bromide (cationic modifier - phase |
| | transfer agent) |
| OA | Oleic acid (negative charge modifier) |

### Naming Convention

In relation to the figures and notation used in the following text, the following is used: Constituent_lipid-modifier_lipid-drug, e.g. MO-DOTAP-1 NOVO—meaning Monololein as the main lipid, additionally modified by DOTAP, and encapsulating 1 mol % of novobiocin as the active agent.

### Plot/Data/Experiment Conventions

Concentrations are in units of ug/ml and are the concentration of the drug in use. Viability is usually assessed by the percentage inhibition for cell growth against varying concentrations. A higher number equates to more dead bacteria. In some instances, viability has been assessed by colony forming unit (CFU) counts. A lower number equates to more dead bacteria.

Materials

Monoolein (MO) (97%, Sigma), Pluronic F127 (Sigma), 1,2-dioleoyl-sn-glycero-3-phosphocholine (DOPC) (99%, Avanti), 1,2-dioleoyl-sn-glycero-3-phosphoethanolamine (DOPE) (99%, Avanti), 1,2-dioleoyl-sn-glycero-3-phospho-(1′-rac-glycerol) (DOPG) (99%, Avanti), 1,2-dioleoyl-3-trimethylammonium-propane (DOTAP) (99%, Avanti), TOAB (99% Sigma), 1,2-dioleoyl-sn-glycero-3-phosphoethanolamine-N-(Cyanine 5) (Cy-5), 1,2-dioleoyl-sn-glycero-3-phosphoethanolamine-N-(lissamine rhodamine B sulfonyl) (99%, Avanti), Octadecyl rhodamine chloride (R18) (Thermofisher), Ethanol (AR), Isopropanol (AR) were used as received without further purification. Peptidogylcan extracts (from S. Aureus) and lipoteichoic acid (from S. Aureus) were used as received by preparing a 1 mg/ml dispersion. Rifampicin (98%, Sigma), Fusidic Acid (98%+, Sigma), Ampicillin (Sigma), Piperacillin (Sigma), Gentamicin (Sigma), Vancomycin (Sigma), Filipin (Sigma), Amphotericin B (Sigma), Benzyl Penicillin (Sigma), Meropenem (Sigma), Clarithromycin (Sigma), Novobiocin (Sigma), Ethambutol (Sigma), Isoniazid (Sigma), Streptomycin (Sigma) and Pyrazinamide (Sigma).

### McFarland 0.5 Barium Sulfate Turbidity Standard:

To standardize the inoculum density, a BaSO₄ turbidity standard is used (0.5 McFarland standard). The procedure consists of the following steps:
(1) Prepare this turbidity standard by adding 0.5 mL of 0.048 mol/L BaCl2 (1.175% w/v BaCl2×2H2O) to 99.5 mL of 0.18 mol/L H2SO4 (1% v/v).
(2) Verify the correct density of the turbidity standard by using a spectrophotometer with a 1-cm light path and matched cuvette to determine the absorbance. The absorbance at 625 nm should be 0.08 to 0.10 for the 0.5 McFarland standard.
(3) Distribute 4 to 6 mL into screw-cap tubes of the same size as those used in growing or diluting the broth culture inoculum.
(4) Tightly seal these tubes and store them in the dark at room temperature.
(5) Vigorously agitate this turbidity standard on a mechanical vortex mixer just before use.
(6) Replace standards or recheck their densities three months after preparation.

### Establishing Effects of Active on Particles and Optimisation of Particle Design

Particle preparation: lipid components (e.g. monoolein, 50 mg) are mixed with active agent (e.g. Cy5, 0.1 mg) in ethanol (0.5 ml). The solution is dried in a vacuum oven for at least 12 hours. The homogeneous lipid mixture is hydrated by a solution of stabilizer (e.g. 0.5% wt F-127 block copolymer). The mixture is dispersed by probe sonication (Branson ultrasonifier 250, 50% Duty Cycle, 5 min), resulting in a 5 wt % dispersion of cubosomes. Variations in lipid composition and encapsulated cargo may be introduced in the lipid mixing stage. Stabilizers and altered lipid components may be used in efforts to minimize any undesired phase transitions, release profiles or general reductions in colloidal stability outlined below.

Colloidal properties of the particles will be characterized prior to application. Particle sizing and zeta potential will be determined by dynamic light scattering (DLS). The internal nanostructure of the particle will be determined through SAXS. This characterisation will be performed for both the neat particles and each encapsulated formulation. The particles require low free drug release rates before engaging the pathogen target. Release of the encapsulated drug will be determined from dialysed samples using UV-absorbance measurements under infinite sink conditions. Response to biological media will be determined by repetition of the colloidal and release experiments in the presence of buffers (e.g. PBS), culture media (DMEM), and LB broth to provide context against routine lab experiments, as well as solutions containing serum proteins to resemble in-vivo conditions. The influence of specific proteins will be subsequently determined. Protein characterisation after exposure to biological media, proteins bound to the particles will be screened by SDS page gel electrophoresis using at least 3 repeats per particle/environment combination. Isothermal titration calorimetry (ITC) will be used to quantify the affinity of individual biomolecules to the particles. A general screening uses 2 μL per 360 s however injection volumes and wait times will be optimized against respective heat signatures. Influence of concentration will be screened across (10-100 μM). ITC will be repeated at temperatures outlined in Aim 1 and repeated at least three times per target. Dilution experiments will be performed as controls.

General procedure: Cubosome/hexosomes were prepared by mixing lipids e.g. monoolein (totalling ˜50 mg) in ethanol (1 ml). For inclusion of charge, DOTAP or TOAB was included in the lipid mixing phase to yield 1 mol %, e.g. (MO 49.5 mg, DOTAP 0.93 mg). For drug loading the antibiotics such as Rif (3.46 mg) and Fus (2.17 mg) were included in the lipid mixing phase, respectively for samples MO-DOTAP-3Rif and MO-DOTAP-3Fus. The solution was subsequently dried in a vacuum oven for at least 12 hours. The homogeneous lipid mixture was hydrated by a solution of F-127 (1 mL, 5 mg/mL). The mixture was dispersed by probe sonication (Branson ultrasonifier 250, 50% Duty Cycle, 3 s on 5 s off, 5 min total duration), resulting in a ˜5 wt % dispersion of cubosomes.

Example DOPE loadings: MO-10PE-1DOTAP-monoolein (40 mg), DOPE (9.91 mg), DOTAP (0.93 mg), MO-20PE-1 DOTAP-monoolein (32.5 mg), DOPE (16.7 mg), DOTAP (0.93 mg), MO-30DOPE-1TAP-monoolein (26 mg), DOPE (24 mg), DOTAP (0.93 mg), MO-40DOPE-1TAP-monoolein (21.5 mg), DOPE (28.2 mg), DOTAP (0.93 mg), MO-1 DOTAP-1 Rif-monoolein (49.5 mg), DOTAP (0.93 mg), Rifampicin (1.15 mg), MO-1 DOTAP-2Rif-monoolein (49.5 mg), DOTAP (0.93 mg), Rifampicin (2.30 mg), MO-1 DOTAP-3Rif-monoolein (49.5 mg), DOTAP (0.93 mg), Rifampicin (3.46 mg), MO-1 DOTAP-4Rif-monoolein (49.5 mg), DOTAP (0.93 mg), Rifampicin (4.62 mg), MO-1 DOTAP-1 NOVO-monoolein (49.5 mg), DOTAP (0.93 mg), novobiocin (0.86 mg), MO-1 DOTAP-3NOVO-monoolein (49.5 mg), DOTAP (0.93 mg), novobiocin (2.58 mg), MO-1 DOTAP-5NOVO-monoolein (49.5 mg), DOTAP (0.93 mg), novobiocin (4.29 mg), MO-1 DOTAP-1 PIP-monoolein (49.5 mg), DOTAP (0.93 mg), piperacillin (0.72 mg), MO-1 DOTAP-3PIP-monoolein (49.5 mg), DOTAP (0.93 mg), piperacillin (2.17 mg), MO-1 DOTAP-5PIP-monoolein (49.5 mg), DOTAP (0.93 mg), piperacillin (3.63 mg).

MTS Based Cell Viability Assay on E. coli O157:H7

Pure culture of E. coli O157:H7 was obtained from the microbial culture collection of the RMIT University, Australia. Bacterial strain was maintained in nutrient broth (NB) and nutrient agar (NA) (Sigma-Aldrich, Australia). Syringe filter (0.45 μm, PES) was purchased from Millipore, Australia. Sterile Technoplas Petri Dish was purchased from Interpath Services, Australia. MTS assay kit (Promega CellTiter 96 Aqueous One Solution) was purchased from Promega.

### Cell Viability Assay

An MTS assay kit was used to determine cell viability, based on the mean value for the activity of mitochondrial succinate dehydrogenase.

The E. coli O157:H7 and Pseudomonas aeruginosa (ATCC 2835) bacterial cells were cultured in nutrient broth (NB) medium within a 10 mL sterile plastic screw cap centrifuge tube with an optical density (O.D600) of 0.5-0.6 and grown at 37° C. with shaking at 150 rpm.

The cell viability determination assay against E. coli O157:H7 and Pseudomonas aeruginosa (ATCC 2835) bacterial strain was performed in duplicate for all sets of samples. Each set containing either neat antibiotics or antibiotics loaded nanoparticles. Neat antibiotic stock solution was prepared in dimethyl sulfoxide (DMSO). 1% bacterial inoculum was added to the positive control contained with DMSO (1%) and negative control contained 50 μg/mL MO-based cationic (DOTAP 1 mol %) lipid nanoparticles. Different antibiotic loaded nanoparticles were filtered using sterile millex-GP syringe filter (0.45 μm, PES, Millipore) and used for the experiments. Free antibiotics (stock 5 mg/mL in DMSO) and antibiotic loaded lipid nanoparticles with intended concentrations were added at the time of inoculation to the 5 mL of culture (with 1% inoculum) medium and incubated for 20-24 hours, at which time the bacterial growth reaches the stationary phase at 37° C. with shaking at 150 rpm.

From each sample 100 μL cell culture medium was taken and dispensed in the 96 well plate. Bacterial cell viability was measured by using MTS assay kit. 10 μL of MTS solution was added per 100 μL of sample and incubated for 2 hours at 37° C. A microplate reader (SpectraMax, Molecular Devices) was used to measure the absorbance at 490 nm. The measured absorbance from the control sample was set to 100% cell viability. All other sample data were therefore adjusted relative to this value.

CFU Based Cell Viability Assay on E. coli O157:H7

Minimal inhibitory concentration (MIC) determination assay. Pure culture of E. coli O157:H7 was obtained from the microbial culture collection of the RMIT University, Australia. The bacterial strain was maintained in nutrient broth (NB) and nutrient agar (NA) (Sigma-Aldrich, Australia). The MIC determination assay against E. coli O157:H7 was performed in duplicate in all sets of samples, with each set containing either free rifampicin (or fusidic acid) or rifampicin (or fusidic acid) loaded nanoparticles of the disclosure. For each replicate, 50 μL of an overnight culture of E. coli was diluted into 5 mL of nutrient broth (NB) and grown at 37° C. with shaking at 150 rpm to an optical density (O.D600) of 0.5-0.6. Free rifampicin and fusidic acid stock solutions were prepared in DMSO. Positive control contained 1% bacterial inoculum with DMSO (1%) and negative control contained 50 μg/mL MO-based cationic (DOTAP 1 mol %) lipid nanoparticles. Rifampicin and fusidic acid loaded nanoparticles were filtered using sterile millex-GP syringe filter (0.45 μm) before use.

The concentration of drugs before and after filtration was evaluated with UV-Vis spectroscopy. A 4 mg/ml stock solution of rifampicin was prepared in ethanol. Ten standard serials dilution solutions with a concentration of 0, 2, 1, 0.5, 0.25, 0.125, 0.0625, 0.03125, 0.015, 0.0078 and 0.0039 mg/ml were prepared in ethanol and the absorbance at 340 nm was plotted against the relative concentration to obtain the standard calibration curve. 50 μL of rifampicin (3 mol % or 3.5 mg/mL) loaded MO-based cationic (DOTAP 1 mol %) lipid nanoparticles were dissolved in 950 μL of ethanol solution before and after filtration. The rifampicin concentration of both the nanoparticle samples (before and after filtration) was measured from the standard curve of rifampicin. It was found that after filtration the rifampicin concentration was ˜5% lower than the unfiltered sample. Free rifampicin (stock 5 mg/mL in DMSO) and rifampicin (3 mol % or 3.5 mg/mL) drug loaded MO-based cationic (DOTAP 1 mol %) lipid nanoparticles were added at the time of inoculation at a concentration ranging from 0.05-5 μg/mL to the 5 mL of culture medium. Bacterial growth was measured by reading the absorbance at 600 nm as well as by determining colony forming units (CFU/mL) after 24 hours of incubation, at which time the bacterial growth reaches the stationary phase. The minimal inhibition concentration (MIC) was defined as the minimum concentration of free drug or nanoparticles that inhibited any growth against the bacterium on the nutrient agar plate (NA) beyond 90%.

### Fungal Cell Viability Assay

Candida albicans (ATCC10251) organism was sub-cultured from sterile vials onto Sabouraud dextrose agar or potato dextrose agar and passaged to ensure purity and viability. The incubation temperature throughout was 35° C.

The inoculum was prepared by picking five colonies of ˜1 mm in diameter from 24-hour old cultures of Candida albicans. The colonies were suspended in 5 mL of sterile 0.145-mol/L saline (8.5 g/L NaCl; 0.85% saline). The resulting suspension was vortexed for 15 seconds and the cell density adjusted with a spectrophotometer by adding sufficient sterile saline to increase the transmittance to that produced by a 0.5 McFarland standard (given below) at 530 nm wavelength.

This procedure will yield a fungal cell stock suspension of 1×106 to 5×106 cells per mL. A working suspension is made by a 1:100 dilution followed by a 1:20 dilution of the stock suspension with RPMI 1640 broth medium, which results in 5.0×102 to 2.5×103 cells per mL.

The cell viability determination assay against Candida albicans strain was performed in duplicate for all sets of samples. Each set containing either neat antibiotics or antibiotics loaded nanoparticles. Neat antibiotic stock solution was prepared in dimethyl sulfoxide (DMSO). 1% of cell stock suspension was added to the positive control contained with DMSO (1%) and negative control contained 50 μg/mL MO-based cationic (DOTAP 1 mol %) lipid nanoparticles. Different antibiotic loaded nanoparticles were filtered using sterile millex-GP syringe filter (0.45 μm, PES, Millipore) and used for the experiments. Free antibiotics (stock 5 mg/mL in DMSO) and antibiotic loaded lipid nanoparticles with intended concentrations were added at the time of inoculation to the 5 mL of culture (with 1% of cell stock suspension) medium and incubated for 24-30 hours, at which time the fungal cell growth reaches the stationary phase at 35-37° C.

From each sample 100 μL cell culture medium was taken and dispensed in the 96 well plate. Fungal cell viability was measured by using MTS assay kit. 10 μL of MTS solution was added per 100 μL of sample and incubated for 2 hours at 37° C. A microplate reader (SpectraMax, Molecular Devices) was used to measure the absorbance at 490 nm. The measured absorbance from the control sample was set to 100% cell viability. All other sample data were therefore adjusted relative to this value.

### Results

Interaction with Gram-Positive Bacteria

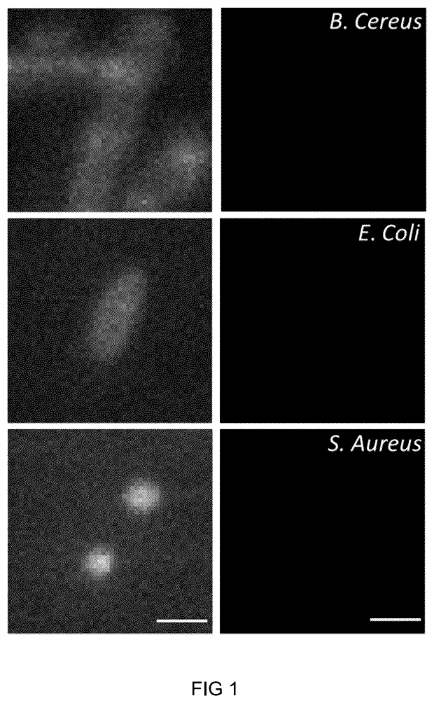

Cubosome uptake was evaluated into two gram-positive bacterial species, Bacillus cereus (B. cereus) and Staphylococcus aureus (S. aureus). The outer most layer of peptidoglycan is estimated to be approximately 50 and 23 nm thick for these species, respectively. Total internal reflection fluorescence microscopy (TIRF) was used to reveal the cubosome interactions in-situ. The bacterial cells could be observed by their intrinsic fluorescence from excitation at λₑₓ=405 nm, shown in blue. Meanwhile, the cubosomes were fluorescently tagged by phospholipid dye 18:1-Cy5 (λₑₓ=647 nm) shown in red. The bacteria themselves demonstrate negligible fluorescence at this wavelength (λₑₓ=647 nm). Representative fluorescence images of bacteria in the absence of fluorescent tags are shown in FIG. 1.

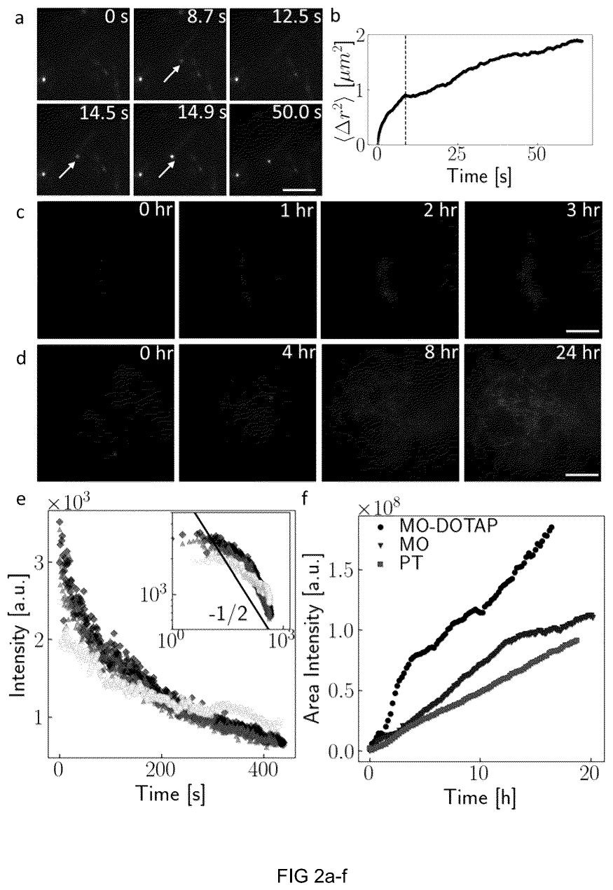

A time-lapse sequence showing the uptake of fluorescently tagged cubosomes into B. cereus is shown in FIG. 2a. From the time lapse in FIG. 2a, a transient cubosome in solution is highlighted by the white arrow. The cubosome, visible as the bright circular spot, was observed to move throughout the solution between frames at t=8.7 s and 14.5 s before adhering to the B. cereus surface at t=approximately 14.9 s. The mean squared displacement (Δr²) was determined through particle tracking and shown in FIG. 2b. The sudden deviation in slope at approximately 8.7 s (indicated by the dashed line), is consistent with the attachment of the cubosome to the surface of the bacterium. Note that in the displacement data, t=0 corresponds to the appearance of the cubosome (nearest to frame 8.7 s in the time-lapse in FIG. 2a). Once attached, the cubosome remained on the surface of the bacterium, even while the cubosome moved in solution. This is also shown by the displacement tracking shown in FIG. 2b where once attached, the reduced gradient indicates motion of a significantly larger colloid, i.e. the bacterium-cubosome complex. The time-lapse in FIG. 2c shows that over a longer time (approximately 3 hours), cubosomes were observed to consecutively attach to the surface of the blue bacteria (λₑₓ=405 nm) as the bright red spots (λₑₓ=647 nm). The cubosomes demonstrated long term stability with maintained size and shape over a period of many minutes.

The extended time-lapses in FIG. 2c-d show that after many hours, the initially localized source of fluorescence from the cubosome has gradually spread covering the entirety of the B. cereus cell. This is qualitatively observed in the time-lapses by the steady increase in red signal. This behaviour has been quantified in FIG. 2e-f. The fluorescence intensity (I) of individual cubosomes (FIG. 2e) decayed exponentially with time. Plotting the data on a log scale revealed two distinct regimes: the first approximately 15 s characterised by a slope of − 1/9 and a dominant slope, between approximately 15 s and 400 s, of −½, indicating an intensity decay where I˜t^{1/9} or I˜t^{1/2} for each regime, respectively. This longevity is in stark contrast to the uptake behaviour observed in eukaryotic cells, where cubosomes appear to fuse within 10 s. It is expected that in the early stages of attachment the cubosome may be wetting the surface i.e. the outer peptidoglycan layer of the B. cereus. The dominant scaling observed for the latter portions (15 s onwards), correlates with what would be expected for diffusion of a point source release in 1-dimension, Cₘₐₓ·t^{0.5d} where Cₘₐₓ is the maximum concentration at time t, and d is the dimensionality. This can be rationalized conceptually by the cubosome constituent lipids diffusing inward through the thick peptidoglycan layer.

The cumulative effect of many cubosomes interacting with the bacterium is reflected in the fluorescence intensity across the entire cell which is shown to steadily increase (FIG. 2f). The increase in fluorescence intensity was observed across all cubosome compositions. The magnitude and rate of fluorescence intensity increase is reasonably similar between MO and PT however there was a marked acceleration and greater magnitude for positively charged cubosomes. For example, following 3 hours of incubation, the intensity observed for MO-DOTAP cubosomes (ζ=+31 mV) was double that of MO cubosomes (ζ=−12 mV). The increase can be attributed to an increased number of successful collisions with the bacterium, promoted by the attractive potential between the negatively charged B. cereus membrane and positive cubosome surface.

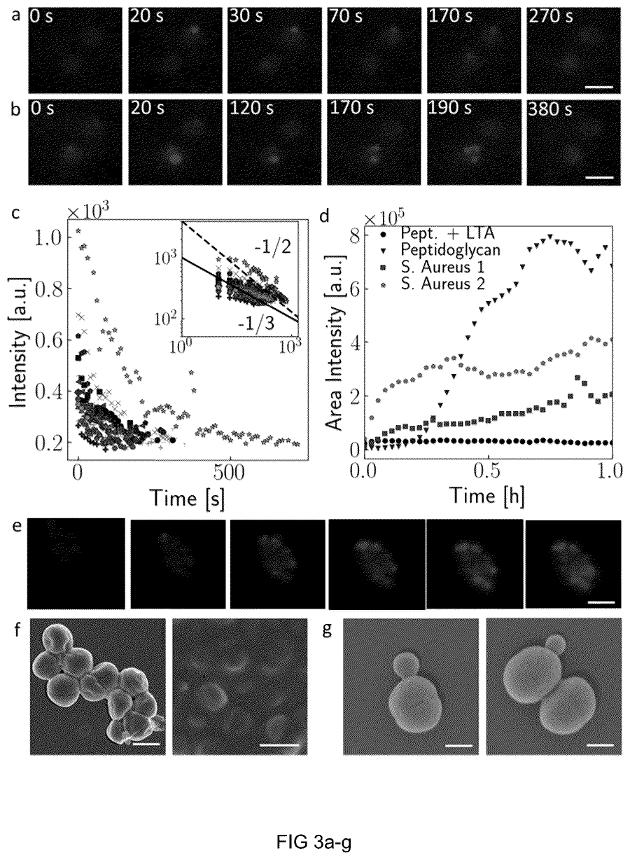

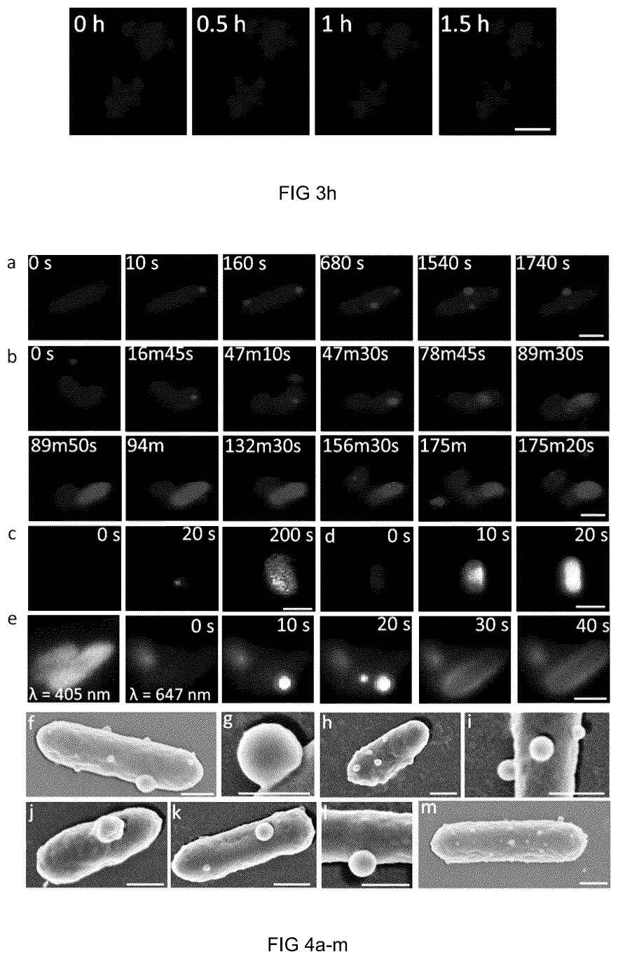

The uptake of cubosomes into S. aureus bacteria is shown in FIG. 3. While the smaller spherical shaped bacteria were somewhat harder to observe, they did, however, remain consistently attached to the surface which allowed for clearer particle tracking. Two representative time-lapses are shown in FIG. 3a-b. In the first series, individual cubosomes can be observed hitting the surface before gradually dissipating over the course of approximately 4 min. In the second series, an increased number of cubosomes was observed, including the simultaneous attachments of three individual cubosomes to the lower bacterium. In this series the cubosomes were clearly observed to land and dissipate over approximately 2-7 min. The time-lapse indicate that the cubosomes generally retain their shape but reduce in size following attachment. The lowermost cubosome in FIG. 3b however shows a clear contraction in the cubosome lateral footprint.

Within the same time-lapse (FIG. 3b), there was some variation in size and duration of attachment which is attributed to the variation in particle size. Presumably larger cubosomes had larger footprints with increased intensity and took a longer time duration to completely decay. The particles appeared to dissipate following 2-10 min. The peak fluorescence intensity with time for individual cubosomes was tracked again, with representative plots shown in FIG. 3c. Within the inset of FIG. 3c, the data on a log scale demonstrates correlation to a slope of approximately −⅓, with some points closer to −½. Interestingly, this scaling is reasonably consistent with that observed for B. cereus. The similarity between scaling for both the gram-positive bacteria suggests that the interaction is dominated by the presence of the outer peptidoglycan layer. The variation in slope between −⅓ and −½ may result from geometry differences. The reduced surface area available of S. aureus relative to B. cereus may even result in interactions between fusing cubosomes. Nevertheless, the dominant scaling observed again correlates to a 1-d point source release of material through the outer layer of peptidoglycan.

A peptidoglycan extract from S. aureus was used to further evaluate the interaction between cubosomes and the outer peptidoglycan layer of the bacteria. Based on the resemblance between the extract and S. aureus themselves, the neat extract provided peptidoglycan shells which retained the shape of the original bacteria. In this case, the resolution of the imaging was somewhat clearer (FIG. 3e) than live S. aureus. The spheroid particles typically existed in planar sheets or stacks at the interface. From the time-lapse (FIG. 3e) many cubosomes were observed to accumulate around the periphery of the cluster. With time the red fluorescence was observed to gradually spread across the cluster domain. In contrast to in-vitro experiments, the cubosomes did not appear to reduce in footprint or intensity.

The mimic was examined in an analogous experiment by including additional cell wall components. In this case, the peptidoglycan extract was incubated with a lipoteichoic acid (LTA) extract (50% w/w LTA:Peptidoglycan). In stark contrast to the previous result, no cubosome attachment to the combined extract was observed. A time-lapse for cubosome attachment to the combined extract is shown in FIG. 3h. The relative uptakes between clusters of S. aureus, peptidoglycan and peptidoglycan+LTA extracts are quantified in FIG. 3d. In general, the S. aureus clusters themselves demonstrated a steady uptake with some peaks attributed to the addition of new cubosomes. This gradual increase is once again reflective of the uptake of cubosome material to the S. aureus, in correlation with earlier results shown by B. cereus. In comparison, the peptidoglycan and peptidoglycan+LTA extracts demonstrated an enhanced and hindered uptake, respectively. It appears that the presence of LTA, which is extensively found in the peptidoglycan layer, hinders cubosome attachment although the exact mechanism for this has not been elucidated.

To investigate the attachment with higher resolution, SEM was performed on the peptidoglycan extracts and S. aureus (FIG. 3f, g). Again, the extract was similar in geometry to fixed S. Aureus. The SEM images show that after treatment with cubosomes, the roughness between extract bodies was clearly drowned out indicating that cubosome material had spread and engulfed the extract (FIG. 3f). In comparison, no noticeable contrast in geometry was observed between treated and non-treated S. Aureus bacteria. This may reflect a decomposition of the particles by the live bacterium, or perhaps the timing of the cubosome interaction, as SEM only captures a single snapshot in time. For cubosome treated then fixed S. aureus, the SEM revealed bimodal distribution of neat bacteria and those decorated by smaller circular features on the edge of the bacterium. Two representative images are shown in FIG. 3g, which highlight the spherical feature external to the cell. The size of the feature (290 and 220 nm lateral diameter in left and right panels) is consistent with the size of the cubosomes. In addition, the location is consistent with the uptake locations observed during TIRF measurements. The apparent contact angle between the spherical feature and the S. aureus bacterium in FIG. 3g are approximately 132° and 120°, respectively. The high contact angle is consistent with the minimal spreading of the cubosome in TIRF measurements, although the angle may have been altered by physical treatments during fixing. The physical stability of the cubosome in vacuum is attributed to crosslinking of the unsaturated bonds and hydroxyl in MO by Osmium and glutaraldehyde. Any detail of the internal structure will not be resolved by SEM, particularly given the 5 nm sputter coating. It is noted that polymerisation and negative staining has previously allowed TEM of the mesostructure. These results indicate the difficulty in approaching the treatment of gram positive bacteria.

Interaction with Gram-Negative Bacteria

The interactions with gram-negative bacteria were examined using Escherichia coli (E. coli). Like S. aureus, E. coli remained well adhered to the interface. From the time-lapse shown in FIG. 4a, the cubosomes were observed attaching consecutively over approximately 30 min. Once attached, the cubosomes appeared to gradually fade and contract, largely behaving as described earlier for gram-positive species. While these single cubosome interactions were most common, interactions involving multiple cubosomes were also observed, as seen in the time-lapse in FIG. 4b which spans a larger period of approximately 3 hours. Between 0 and approximately 47 min a single cubosome was observed to adhere to the bacterial surface. Following this, a secondary adsorption was observed on top of the previously adhered cubosome. In the frames at approximately 90 min, a new cubosome was observed to land and prompt fluorescence to diffuse throughout a significant portion of the cell. By the 94 min frame, the entire cell was brightly fluorescent at the cubosome wavelength. In the latter frames of this sequence, this process is shown to repeat (approximately 175 min). These sequences suggest that cubosome fouled regions of the bacteria are favourable landing sites for new cubosomes, possibly enabled by the inclusion of fusogenic lipids into the cell membrane. Furthermore, that the cubosome material can rapidly spread through the entirety of the E. coli bacterium.

By examining uptake on a single channel (λₑₓ=647 nm) the incidents of rapid uptake can be more clearly resolved. The brief series in FIG. 4c highlights this for MO cubosomes. In the upper panel a small circular feature is resolved to be approximately 100-200 nm in diameter at 20 s. Suddenly, after 3 minutes, this feature appeared to induce a burst of fluorescence across the entire cell body. An analogous event is shown for MO-DOTAP cubosomes in FIG. 4d, where the cubosome attachment can be inferred by the centralized bright point at the centre right of the cell body. As described above, for gram-negative bacteria the cubosome will first interact with the outer plasma membrane rather than the peptidoglycan layer. In this sense, the interaction between cubosomes and gram-negative bacteria draws more parallels to the interaction with mammalian membranes. The rapid spread of material may reflect the ability of the cubosome material to fuse with the outer membrane. Transfer of material via the plasma membrane is arguably most clear in FIG. 4e. The initial frame shows the signal channel of λₑₓ=405 nm to highlight the bacterial location. In the following image series (λₑₓ=647 nm) two cubosomes can be observed landing at 10 s and 20 s, respectively. By 30 s, the local fluorescence had dissipated throughout the bacterium. Interestingly, an increased signal was observed at the perimeter of the cell consistent with location of the dye in the lipid membrane. In this series, it is possible that the combination of rapid interaction and a large volume of cubosome material has allowed for the temporal gradient of material in the membrane to be resolved.

SEM images shown in FIG. 4f-m revealed remarkably similar images of cubosome attachment to those shown in FIG. 4a. From the series of images, several distinct spherical asperities can be observed on the surface of the E. coli. The size of the protrusions in the images varies in diameter from approximately 400 to approximately 50 nm. In contact with the E. coli surface it appeared that larger cubosomes exhibited a higher contact angle with the bacterial surface. This is most evident from FIGS. 4g and l where the contact angle appears greater than 90°. In comparison the small cubosomes appear more spread and engulfed into the bacterial surface. From the variation in size it can be inferred that each of the cubosomes has been captured at various stages of internalization to the bacterium, effectively frozen by the fixing process.

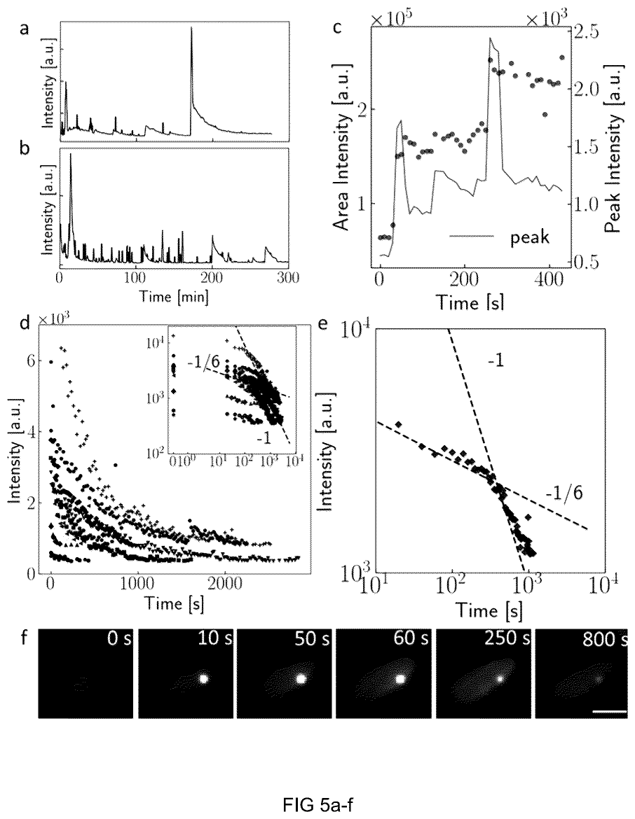

The rate of uptake by an individual E. coli bacterium was assessed by quantifying the fluorescence intensity with time. The number of individual cubosome uptake events can be observed in FIG. 5a-b, which are representative results for MO and MO-DOTAP cubosomes, respectively. Many peaks in intensity are observed which can be attributed to the attachment of cubosomes to the cell. Between the two plots it is immediately clear that MO-DOTAP results in an increased frequency of peaks attributed to cubosome attachment. This is in general agreement with results shown earlier for B. cereus and previous results for liposomes where the hindrance has been minimized by the incorporation of positive charge to the liposome. Focusing on a narrower time duration in FIG. 5c, the red curve shows the peak intensity curve with two prominent peaks from cubosome attachment(s). The grey points show the fluorescence intensity recorded for the area of the entire cell. Following each cubosome attachment event, the peak intensity decays while there is a stepwise increase in the area intensity. The combination of these shows the transfer of imparted fluorescence from the cubosome to the bacterium. Moreover, this result clearly demonstrates the quantized delivery of loaded components from the cubosome.

By tracking the intensity with time for individual cubosomes attached to E. coli, an additional difference was observed to that of the gram-positive species. While still displaying an exponential decay, on a log scale, two regimes were observed, as indicated by the two slopes −⅙ and −1 (FIG. 5d). One representative result is plotted in FIG. 5e with a corresponding time series of images in FIG. 5f. These two slopes suggest that the interaction of a cubosome with E. coli occurs in two stages. Given homogeneous dispersion of the fluorophore within the cubosome, the intensity Iₘₐₓ scales with cubosome size (R) and a mass (m) balance can be derived by \dot{m}=ρR²\dot{R}=Dhδc which is driven by the concentration gradient δc as the cubosome diffuses into a 2D lipid bilayer of height h. The evolution of δc with time scales ˜t^{3/2} based on 2D diffusion intertwined with an expanding diffusive length (half of the contact footprint diameter of the particle,

$$L / 2 ) ; δ c = \frac{{c}_{max} - {c}_{L / 2}}{L / 2} ∼ \frac{1 / t}{\sqrt{t}} = {t}^{- 3 / 2} .$$

The integration of the equations arrives at a final intensity scaling of Iₘₐₓ˜t^{1/6} which provides a good agreement with the data obtained in the early stages of the experiments. The fusion can qualitatively be observed by gradual reduction in spot size of the nanocarrier and the clear gradient in intensity around the cell perimeter in FIG. 5f.

$${I}_{max} ∼ R ∼ \sqrt[3]{\frac{{Dht}^{- 1 / 2}}{ρ}} ∼ \sqrt[3]{\frac{Dh}{ρ}} {t}^{- 1 / 6}$$

At the later stage, the slope gradually transitions to a value of −1 indicating a scaling of I˜t¹. This scaling suggests that the cubosome is delivering material as point source, effectively in two-dimensions. The two-step process can be inferred as (1) fusion into the plasma membrane and (2) continuous diffusion through the cell wall. A mass balance between the fusing lipids and bacteria can be built as \dot{m}=Fₘ+F_{w}, where Fₘ and F_{w} represent the diffusion of lipid in outer plasma membrane and inner cell wall, respectively. Given that cubosomes initially meet the plasma membrane, which drives the concentration gradient in the plasma membrane, fusion dominates at the early stage, Fₘ>>F_{w}, and F_{w} can be ignored. Then, as the lipid concentration in the membrane gradually increases, lipid transfer through the inner wall into the interior of the cell is enhanced. As shown in FIG. 5d, for the shorter-lived interactions, the fusion phase dominates. Then in the latter stages, the intensity decays as the material transitions from the outer membrane through the inner peptidoglycan layer, with the cubosome acting as a point-source.

It would be expected that antimicrobials including conventional antibiotics (e.g. beta-lactams, ansamycins) or antimicrobial peptides may be delivered in a similar fashion to the phospholipid dye studied here. Assuming no leakage of the encapsulated material, the delivery should be quantized by the number of cubosomes attached. Delivery to gram-positive bacteria scaled with the exponent −½, is consistent with 1-D point release. For gram-negative bacteria, the two-stage process involved initial fusion with exponent −⅙ followed by release of the cargo as a point source in 2-D with exponent −1, suggesting that delivery can be more effective than in gram-positive species. This is further supported by the observation that the delivered compound can rapidly spread throughout the entirety of the gram-negative bacterium and suggests that the plasma membrane of gram-negative bacteria aids the integration of the cubosome material and cell wall. The ability to circumvent this outer membrane may prove vital in the delivery of therapeutics to increasingly resilient gram-negative species. Moreover, the inclusion of fusogenic lipids which lower energetic barriers to fusion should further promote this process.

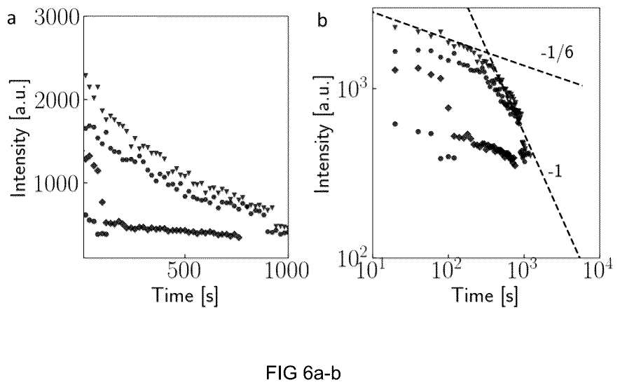

The intermittent occurrences of rapid fluorescence spreading demonstrated in FIG. 4c-d, caused deviation from the two-stage model. A subset of intensity versus time curves for cubosomes which exhibited a rapid spread during their attachment is shown in FIG. 6. In general, the intensity curves follow the scaling previously shown in FIG. 6a-b, however, each curve here also has a steep drop in intensity marked by an arrow in the plot. This drop corresponds to the point in time at which the localization of fluorescence expanded to the entire bacterium. The time taken for the drop to occur was sporadic and did not occur for all cubosomes. In each case, the peak fluorescence reduced by 40-50% and the drop duration was faster than the usual decay observed (seconds versus minutes). As a result, scaling analysis for this intermittent process was not feasible due to the reduced number of data points; that said, the indicative slope for the drops shown on the log plot varied between approximately −2.13 and 2.33. As these events were irregular and given that entire bacterium became fluorescent, it may be possible that these specific cubosomes (or part of) had traversed the entire cell wall. The deviation from the two-stage transport model may reflect the influence of enzymes secreted by the bacterium. Thorn et al. recently demonstrated that the presence of lipases prompted burst release of hydrophobic cargo.⁸

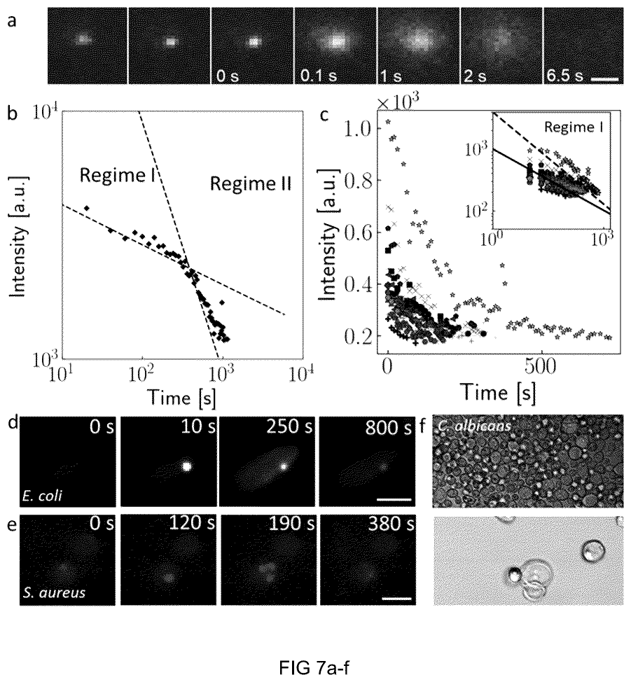

The fusion behaviour between cubic nanocarriers and supported lipid bilayers (SLB) using total internal reflection fluorescence (TIRF) is shown in FIG. 7a. The series shows the fusion of the cubosome material, initially observed as the bright point with fluorescence diffusing radially over approximately 6.5 s. Using the same approach on eukaryotic cells it is possible to directly observe and quantify the kinetics of these interactions. Experiments show colocalization of the cubosomes to bacteria over 12 hours. The results have revealed distinct regimes and differences in transport of cubosome into gram-negative versus gram-positive bacteria, highlighted in FIGS. 7b and 7c, respectively. The snapshots in FIG. 7d-e demonstrate the representative results from in-situ surface sensitive measurements, which allow differentiation between the cubosomes and bacteria, and observation of individual cubosome-bacteria interactions.

In the upper image of FIG. 7f, the rings of red fluorescence around the fungi can be observed. As the fungi undergo mitosis, the fungal membrane deforms to allow the adhered cubosomes to fuse with the plasma membrane. As shown in the lower image in FIG. 7f, broad internalization of the red fluorescence from the cubosomes is observable. It is reasonable to expect the same design criteria for bacteria will be applicable for fungi.

### Delivery of Active Agents

### Novobiocin

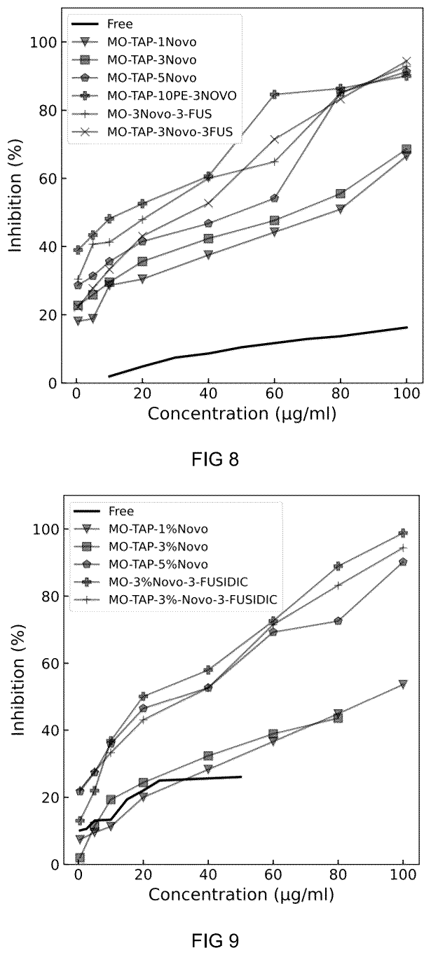

Novobiocin inhibits enzymes involved in DNA synthesis. FIGS. 8 and 9 and Table 2 show the inhibition of P. aeruginosa and E. coli by cubosome formulations containing the antibiotic novobiocin, presented in Table 1. The plots show the inhibition (y-axis) of each formulation against concentration of novobiocin (x-axis). The total concentration is equivalent between each formulation.

Against P. aeruginosa, all encapsulated formulations of novobiocin had significantly more inhibition than the freely dissolved (solid black line).

As the concentration of drug per cubosome (or drug molecules per particle) increased, there was increased inhibition. Covering ranges (1-5 mol %). This result indicates that fewer particles with higher drug loadings are more effective than more particles with a lower drug loading.

For example, at 80 μg/ml, MO-DOTAP-1 NOVO, MO-DOTAP-3NOVO, MO-DOTAP-5NOVO, displayed 51%, 55% and 85% inhibition, respectively. The free antibiotic achieved 13% inhibition.

Inclusion of a secondary drug, fusidic acid, which is an antibiotic/bacteriostatic, that inhibits protein synthesis, also further increased inhibition (plus vs pentagon).

In practice P. aeruginosa typically exhibits high resistance to novobiocin due to limited permeability across the outer plasma membrane. The increased inhibition when encapsulated is attributed to the fusion mechanism demonstrated herein by cubic phase nanoparticles.

Inclusion of ‘fusogenic’ lipids will promote the fusion uptake mechanism. Inclusion of highly curved lipid, DOPE at 10 mol %, increased inhibition by an average of 57% across all concentration ranges. For example, MO-DOTAP-3NOVO and MO-DOTAP-10PE-3NOVO yielded ˜48% and ˜85% at 60 μg/ml, respectively. Novobiocin is not often used in practice due to high inactivation in serum due to binding to serum proteins. In contrast, the inhibition appeared to improve in the presence of serum albumins when novobiocin was encapsulated in cubosomes.

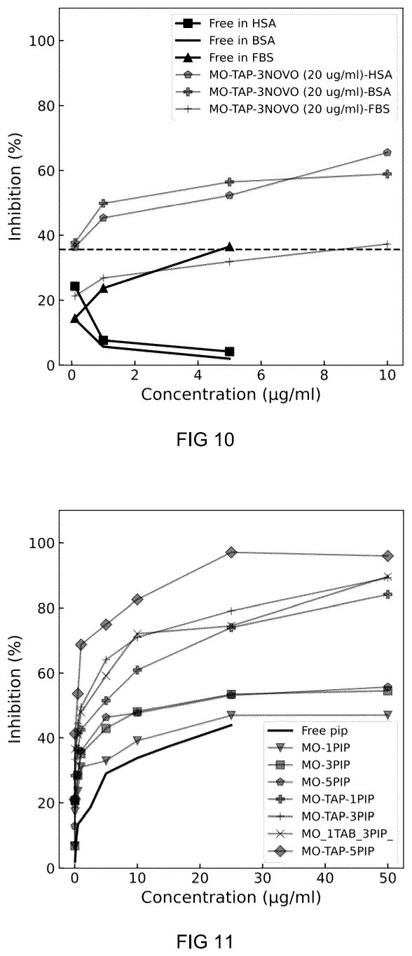

The inhibition was evaluated using MO-DOTAP-3NOVO, in the presence of human serum albumin (HSA), bovine serum albumin (BSA), and fetal bovine serum (FBS). FIG. 10 demonstrates that the encapsulation preserves the antibiotic activity versus the freely dissolved antibiotic (black lines). The dashed line reflects the performance of MO-DOTAP-3NOVO in the absence of serum proteins at equivalent loading (20 μg/ml).

TABLE 1
Formulations containing novobiocin.

| | | Lattice Par. | Avg curvature | Avg |
|---|---|---|---|---|
| Formulation | Phase | (Å) | (nm−1) | CPP |
| MO-DOTAP-1NOVO | Im3m | 143.7 | −0.1596 | 1.168 |
| MO-DOTAP-3NOVO | Im3m | 139.5 | −0.1596 | 1.168 |
| MO-DOTAP-5NOVO | Im3m | 148.6 | −0.1596 | 1.168 |
| MO-3NOVO3Fusidic | Im3m | 148.6 | −0.1596 | 1.168 |
| MO-DOTAP- | Im3m | 145.6 | −0.1596 | 1.168 |
| 3NOVO3Fusidic | | | | |

TABLE 2
CFU counts for e coli after treatment with free
and encapsulated novobiocin in MO-DOTAP-3NOVO. Lower
CFU number indicates improved result or more inhibition.
Final column shows fraction of CFU in cubosome treatment
to CFU count of free antibiotic.

| Conc | CFUs | CFUs | CFU_Cubosome/ |
|---|---|---|---|
| (ug/ml) | Free | Cubosome | CFU_Free |
| 5 | 812 | 467 | 0.58 |
| 10 | 800 | 210 | 0.26 |
| 20 | 476 | 117 | 0.25 |
| 30 | 280 | 65 | 0.23 |
| 40 | 137.5 | 14 | 0.10 |
| 50 | 40 | 0 | 0 |

### Piperacillin

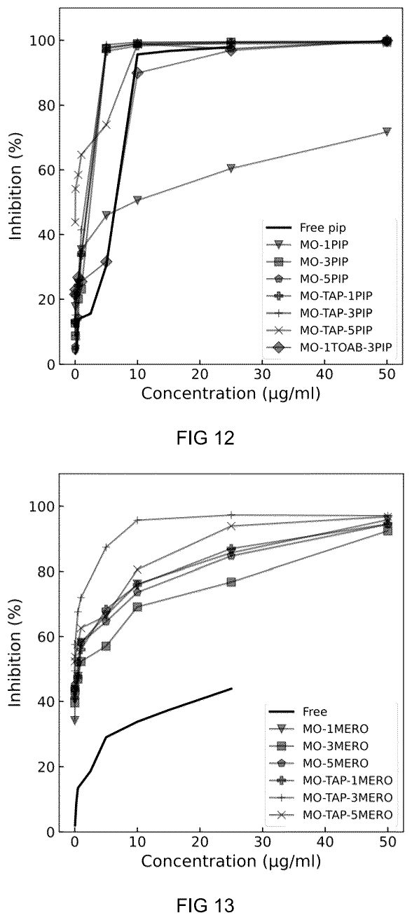

Piperacillin is a beta lactam antibiotic which inhibits crosslinking of peptidoglycan leading to cell rupture. FIGS. 11 and 12 and Table 4 show the inhibition of P. aeruginosa and Ee. coli by cubosome formulations containing the antibiotic piperacillin, presented in Table 3.

Against P. aeruginosa (FIG. 11), all encapsulated formulations of piperacillin had significantly more inhibition than the freely dissolved drug (solid line).

As the concentration of drug per cubosome (or drug molecules per particle) increased, there was increased inhibition.

For example, MO-DOTAP-1 Pip, MO-DOTAP-3Pip, MO-DOTAP-5Pip achieved 52%, 59% and 75%, respectively.

The incorporation of positive charge by inclusion of cationic lipid DOTAP greatly increased the inhibition. This can be observed by the groupings of particles with cationic properties with ˜80%+ inhibition (diamond, cross, plus) and without cationic lipids with ˜50% (inverted triangle, pentagon, square).

Similar inhibition was achieved by inclusion of TOAB as the cationic moiety (cross vs plus).

Against E. coli (FIG. 12), the same trends were observed. Encapsulated piperacillin exceeded performance of the free antibiotic when the encapsulated concentration was above 1 mol %.

Table 4 demonstrates the inhibition for piperacillin against E. coli when encapsulated in cubosomes of distinct key constituent lipids, monoolein or phytantriol. The performance between formulations was similar.

TABLE 3
Formulations containing piperacillin.

| | | Lattice Par. | Avg curvature | Avg |
|---|---|---|---|---|
| Formulation | Phase | (Å) | (nm⁻¹) | CPP |
| MO-1Pip | Im3m | 144.5 | −0.161 | 1.17 |
| MO-3Pip | Im3m | 153.5 | −0.161 | 1.17 |
| MO-5Pip | Pn3m | 125 | −0.161 | 1.17 |
| MO-DOTAP-1Pip | Im3m | 156.3 | −0.159 | 1.168 |
| MO-DOTAP-3Pip | Im3m | 158.3 | −0.159 | 1.168 |
| MO-DOTAP-5Pip | Im3m | 159.1 | −0.159 | 1.168 |
| MO-TOAB-3Pip | Im3m | 134.7 | −0.159 | 1.168 |

TABLE 4
Percentage inhibition for formulations of MO
and PT cubosomes containing piperacilin at 1
mol % against Pseudomonas aeruginosa.

| Drug conc [ug/ml] | PT-PIP | MO-1PIP- |
|---|---|---|
| 0.01 | 6.85 | 12.32 |
| 0.05 | 12.26 | 17.73 |
| 0.50 | 17.91 | 24.96 |
| 1.00 | 26.27 | 35.3 |
| 5.00 | 39.23 | 45.89 |
| 10.00 | 47.59 | 50.57 |
| 25.00 | 65.05 | 60.43 |
| 50.00 | 72.84 | 71.76 |

### Meropenem

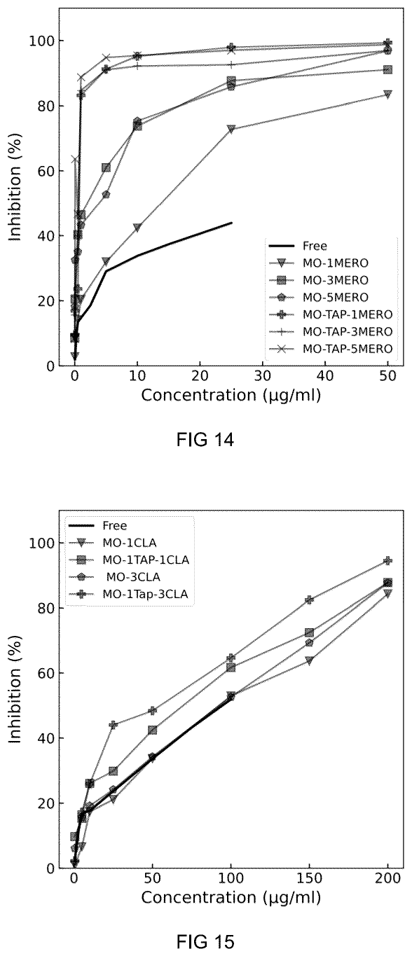

Meropenem is a beta-lactam antibiotic which inhibits cell wall synthesis leading to cell rupture and death. FIGS. 13 and 14 show the inhibition of P. aeruginosa and E. coli by cubosome formulations containing the antibiotic piperacillin, presented in Table 5.

Against P. aeruginosa and E. coli, all encapsulated formulations of meropenem had significantly more inhibition than the freely dissolved drug (solid black line).

As the concentration of drug per cubosome (or drug molecules per particle) increased, there was increased inhibition. In the case of meropenem, this influence appeared saturated between 3-5 mol %.

The incorporation of positive charge by inclusion of cationic lipid DOTAP greatly increased the inhibition. This can be observed by the groupings of particles with cationic properties with ˜80%+ inhibition (diamond-cross-plus) and without cationic lipids with ˜50% (square, triangle, pentagon).

TABLE 5
Formulations containing meropenem.

| | | Lattice Par. | Avg curvature | Avg |
|---|---|---|---|---|
| Formulation | Phase | (Å) | (nm−1) | CPP |
| MO-1mero | Im3m | 142.7 | −0.1613 | 1.17 |
| MO-3mero | Im3m | 145.8 | −0.1613 | 1.17 |
| MO-5mero | Im3m | 147.8 | −0.1613 | 1.17 |
| MO-DOTAP- | Im3m | 146.9 | −0.1596 | 1.168 |
| 1mero | | | | |
| MO-DOTAP- | Im3m | 146.3 | −0.1596 | 1.168 |
| 3mero | | | | |
| MO-DOTAP- | Im3m | 154.7 | −0.1596 | 1.168 |
| 5mero | | | | |

### Clarithromycin

Clarithromycin inhibits protein synthesis. FIG. 15 shows the inhibition of P. aeruginosa and E. coli by cubosome formulations containing this antibiotic, as presented in Table 6.

The incorporation of positive charge by inclusion of cationic lipid DOTAP increased the inhibition. When encapsulated at 1 and 3 mol % within cationic cubosomes, the encapsulated clarithromycin provided increased inhibition of E. coli compared to the freely dissolved antibiotic.

In the absence of cationic lipids, the performance was essentially equivalent between formulations.

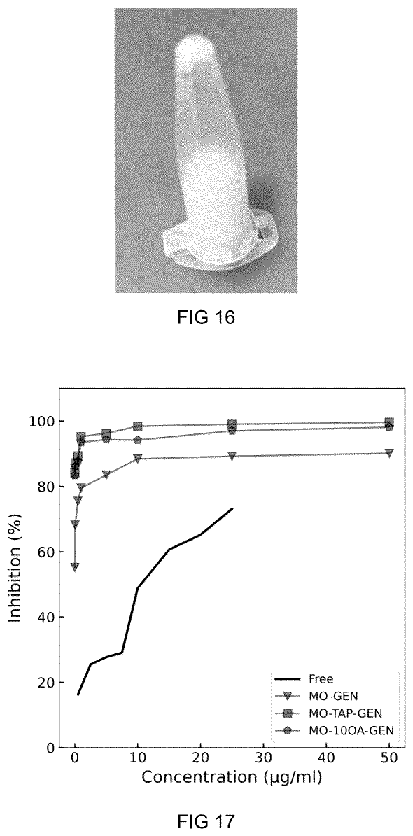

The enhancement was less noticeable with clarithromycin likely due to precipitation of the drug, as pictured in FIG. 16. This may be addressed by additional variations to the formulation to minimise this effect.

TABLE 6
Formulations containing clarithromycin.

| | | Lattice Par. | Avg curvature | Avg |
|---|---|---|---|---|
| Formulation | Phase | (Å) | (nm−1) | CPP |
| MO-1CLA | Im3m | 136.5 | −0.1613 | 1.17 |
| MO-DOTAP- | Im3m | 127.9 | −0.1596 | 1.168 |
| 1CLA | | | | |
| MO-3CLA | Im3m | 136.1 | −0.1613 | 1.17 |
| MO-DOTAP- | Im3m | 123.8 | −0.1596 | 1.168 |
| 3CLA | | | | |

### Gentamicin

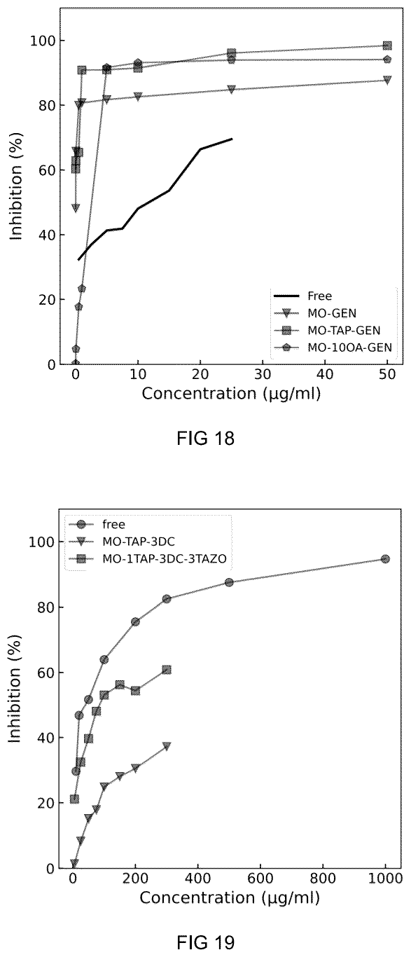

Gentamicin is an aminoglycoside which interrupts protein synthesis and may exhibit some impact on the bacterial cell membrane. FIGS. 17 and 18 show the inhibition of P. aeruginosa and E. coli by cubosome formulations containing this antibiotic, as presented in Table 7.

The inhibition was greatly enhanced when encapsulated, achieving peak inhibitions at significantly reduced concentrations compared to the freely dissolved antibiotic.

A 10-20% increase in inhibition was observed when charged lipid DOTAP or OA was incorporated to the cubosome particle, which increase zeta potential and negative curvature respectively.

The marked contrast can be attributed to the enhanced delivery, coupled with the mode of action of gentamicin, which is a concentration-dependent antibiotic.

It requires a high concentration opposed to a continual presence of a low concentration.

TABLE 7
Formulations containing gentamicin.

| | | Lattice Par. | Avg curvature | Avg |
|---|---|---|---|---|
| Formulation | Phase | (Å) | (nm−1) | CPP |
| MO_GEN_{—} | Im3m | 142.5 | −0.1613 | 1.170 |
| MO_1DOTAP_GEN_{—} | Im3m | 152.3 | −0.159 | 1.168 |
| MO_10OA_GEN_{—} | Pn3m | 92.9 | −0.2702 | 1.193 |

### Dicloxacillin

Dicloxacillin is a narrow spectrum beta-lactam antibiotic which inhibits cell wall synthesis leading to cell rupture and death. Dicloxacillin has limited activity against gram-negative bacteria.

FIG. 19 shows the inhibition of E. coli by cubosome formulations containing the antibiotic Dicloxacillin, as presented in Table 8. The dual encapsulation with tazobactam (a beta lactamase inhibitor) effectively doubled the inhibition (square vs triangle) thereby indicating that even antibiotics not typically amenable to use against gram-negative bacteria may be useful in the approach of the present disclosure when used in combination with a second active agent.

TABLE 8
Formulations containing dicloxacillin.

| | | Lattice Par. | Avg curvature | Avg |
|---|---|---|---|---|
| Formulation | Phase | (Å) | (nm−1) | CPP |
| MO-TAP-3DC | Im3m | 150.8 | −0.159 | 1.168 |
| MO-TAP-3DC- | Im3m | 147.6 | −0.159 | 1.168 |
| 3TAZO | | | | |

### Benzyl Penicillin

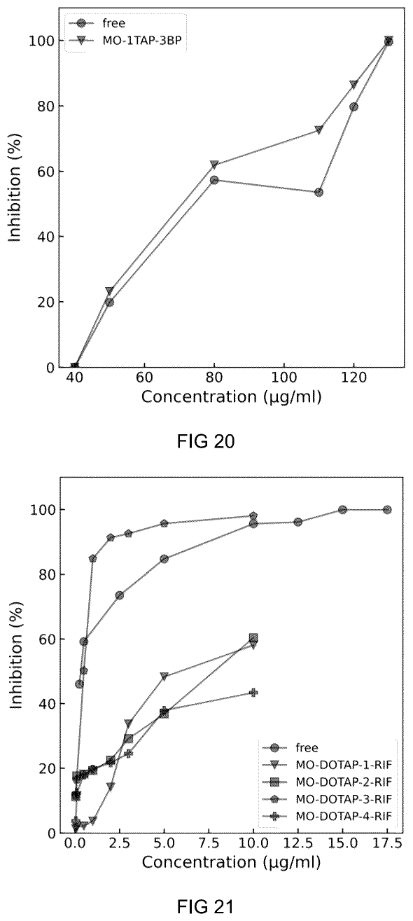

Benzyl Penicillin is a narrow spectrum beta-lactam antibiotic which inhibits cell wall synthesis leading to cell rupture and death. Benzyl Penicillin has activity against gram-negative bacteria due to inherent resistance to beta-lactamase antibiotics. FIG. 20 shows the inhibition of E. coli by cubosome formulations containing the antibiotic benzyl penicillin, as presented in Table 9.

Benzyl Penicillin yielded increases in inhibition when encapsulated at 3 mol % compared to the free antibiotic thereby indicating that simple changes in formulation, such as drug concentration/loading, can be used to improve outcome.

CFU testing revealed 10-84% further reduction in colony counts when encapsulated, generally increasing with concentration (Table 10).

TABLE 9
Formulations containing benzyl penicillin.

| | | Lattice Par. | Avg curvature | Avg |
|---|---|---|---|---|
| Formulation | Phase | (Å) | (nm−1) | CPP |
| MO-DOTAP-1BP | Im3m | 142.775 | −0.159 | 1.168 |
| MO-DOTAP-2BP | Im3m | 148.284 | −0.159 | 1.168 |
| MO-DOTAP-3BP | Im3m | 149.191 | −0.159 | 1.168 |
| MO-DOTAP-5BP | Im3m | 153.405 | −0.159 | 1.168 |
| MO-DOTAP- | Im3m | 161.832 | −0.159 | 1.168 |
| 10BP | | | | |

TABLE 10
CFU counts for E. coli when incubated at
varying levels of free BP and MO-DOTAP-3BP.

| Conc | CFU | CFU | CFU_Cubosome/ |
|---|---|---|---|
| (ug/ml) | Free drug | Cubosome | CFU FREE |
| 40 | 732.5 | 672 | 0.92 |
| 50 | 587.5 | 516.5 | 0.88 |
| 80 | 312.5 | 256 | 0.82 |
| 110 | 340 | 184.5 | 0.54 |
| 120 | 148.5 | 91.5 | 0.62 |
| 130 | 3 | 0 | 0 |

### Rifampicin

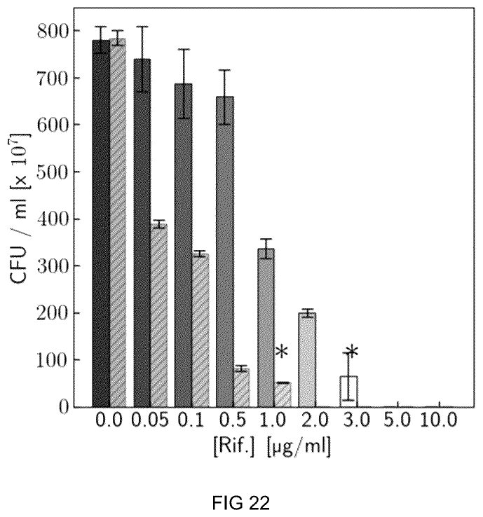

Rifampicin inhibits RNA synthesis. FIGS. 21 and 22 show the inhibition of E. coli by cubosome formulations containing the antibiotic rifampicin, as presented in Table 11.

As the concentration of drug per cubosome (or drug molecules per particle) increased, there was increased inhibition. This result indicates that fewer particles with higher drug loadings can be more effective than more particles with lower individual drug loading. This result also demonstrates encapsulation greatly enhances the effectiveness of the same quantity of drug.

It was noted at loadings of 4 mol %, the inhibition reduces from that of 3 mol %. This loss of functionality is attributed to the loss of ordered structure in the nanoparticle, or more specifically, loss of the fusogenic cubic phase mesostructured due to the higher drug concentration.

TABLE 11
Formulations containing rifampicin.

| | | Lattice Par. | Avg curvature | Avg |
|---|---|---|---|---|
| Formulation | Phase | (Å) | (nm−1) | CPP |
| MO-DOTAP-1 Rif | Im3m | 153.8 | −0.159 | 1.168 |
| MO-DOTAP-2Rif | Im3m | 154.6 | −0.159 | 1.168 |
| MO-DOTAP-3Rif | Im3m | 166.6 | −0.159 | 1.168 |
| MO-DOTAP-4Rif | Lamellar | 46.6 | −0.159 | 1.168 |
| MO_DOTAP- | Im3m | 157.6 | −0.159 | 1.168 |
| 3Rif3Fusidic | | | | |

### Increasing Curvature

The delivery of the antibiotic active agents described herein is proposed, without wishing to be limited by theory, to occur via fusion of the self-assembled nanoparticle and the bacterial membrane. Consequently, inclusion of fusogenic lipids should promote the fusion delivery pathway by further reducing energetic barriers for fusion to occur. For rifampicin this is demonstrated through a series of formulations with increasing concentrations of DOPE, a highly fusogenic lipid (Table 12), and with the presence of a cationic lipid (Table 13).

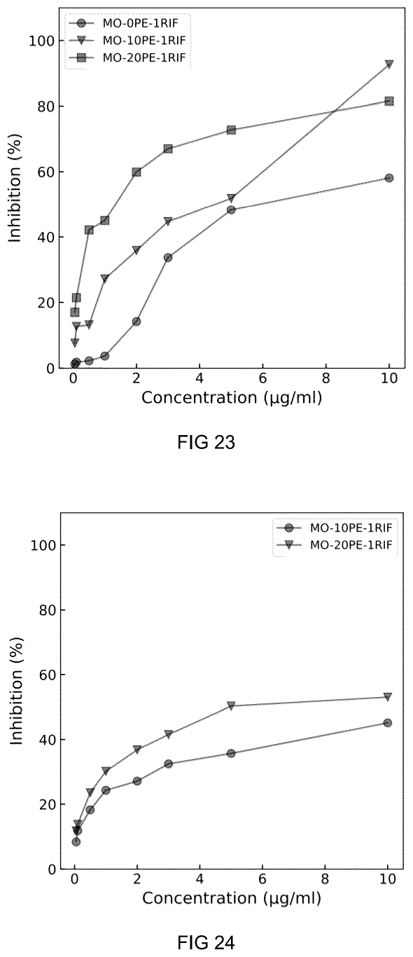

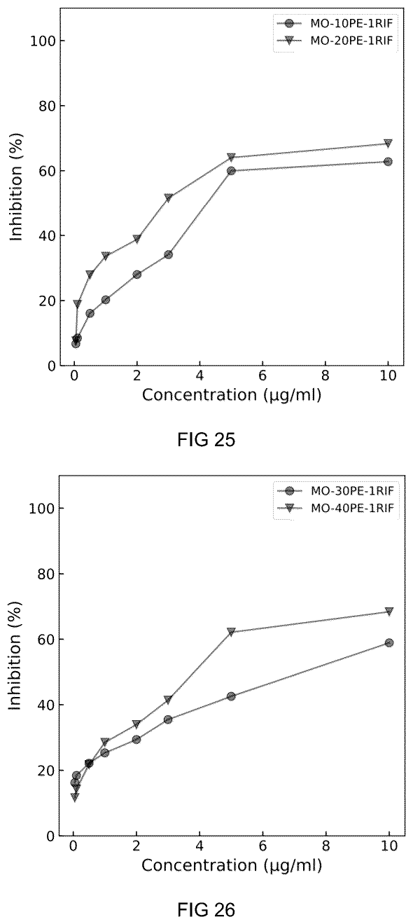

Between 0-20% DOPE (inclusive) the particles are cubic phases. While with 30-40% DOPE the particles display hexagonal symmetry. With increasing DOPE 0-20 (i.e. 0-20%), the lattice parameter of the particles decreased from 146 to 99.6 Å, reflecting an increase in the particle's curvature. The average spontaneous curvature shifted from −0.161 nm⁻¹ to −0.216 nm⁻¹. Simultaneously, the inhibition with 1 mol % rifampicin was observed to increase (FIGS. 22 and 23). For example, at 2 μg/ml the inhibition was 14%, 36%, 60%, respectively.

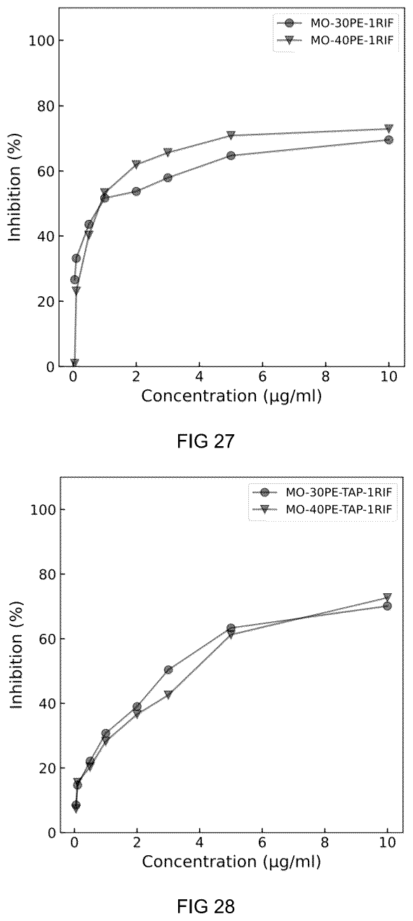

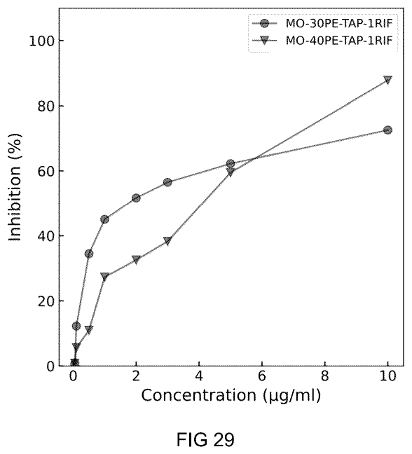

The results demonstrate inclusion of fusogenic lipids increases the effectiveness of cubosomes in the delivery of antibiotics. This trend was also observed with cationic nanoparticles (FIG. 24). Increased inhibition was also observed from formulations with 40% DOPE relative to 30% DOPE (FIGS. 26 and 27). This increase was somewhat less pronounced for cationic nanoparticles (FIGS. 28 and 29).

TABLE 12
Formulations containing rifampicin
with varying degrees of curvature.

| | | | Avg curvature |
|---|---|---|---|
| Formulation | Phase | Lattice Par. (Å) | (nm −1) |
| MO-0PE-1RIF | Im3m | 146 | −0.161 |
| MO-10PE-1RIF | Pn3m | 101.7 | −0.189 |
| MO-20PE-1RIF | Pn3m | 99.6 | −0.216 |
| MO-30DOPE-1RIF | HII | 56.8 | −0.243 |
| MO-40DOPE-1RIF | HII | 57.3 | −0.271 |
| MO-10PE-DOTAP- | Im3m | 140.96 | −0.187 |
| 1RIF | | | |
| MO-20PE-DOTAP- | Pn3m | 97.9 | −0.214 |
| 1RIF | | | |
| MO-30DOPE- | HII | 56.62 | −0.242 |
| DOTAP-1RIF | | | |
| MO-40DOPE- | HII | 56.77 | −0.269 |
| DOTAP-1RIF | | | |

TABLE 13
Formulations containing Rifampicin consisting of monoolein
or phytantriol with varying degrees of oleic acid.

| | | | Avg curvature |
|---|---|---|---|
| Formulation | Phase | Lattice Par. (Å) | (nm −1) |
| MO-10OA-1RIF | Im3m | 121.8 | −0.270 |
| MO-30OA-1RIF | hex | 46.2 | −0.487 |
| MO-50OA-1RIF | Undefined | | |
| PT-10OA-1RIF | hex | 51.2 | −0.270 |
| PT-30OA-1RIF | Hex | 46.3 | −0.487 |
| PT-50OA-1RIF | Mixed phases | 75.9 | |

TABLE 14
Percentage inhibition of E. coli by incubation with MO-OA
particles containing rifampicin with varying concentrations.

| Drug Conc | MO-10OA-1-RIF | MO-30OA-1-RIF | MO-50OA-1-RIF |
|---|---|---|---|
| [ug/ml] | 1 | 2 | 3 |
| 0.05 | 6.86 | 0.75 | 1.57 |
| 0.1 | 9.94 | 6.05 | 4.75 |
| 0.5 | 15.68 | 12.42 | 11.75 |
| 1 | 20.39 | 19.08 | 21.93 |
| 2 | 28.52 | 22.03 | 28.89 |
| 3 | 35.48 | 26.53 | 35.52 |
| 5 | 41.38 | 35.66 | 40.98 |
| 10 | 47.34 | 45.03 | 47.34 |

TABLE 15
Percentage inhibition of E. coli by incubation
with PT-OA cubosomes with varying concentrations.

| Drug Conc | PT-10OA- | PT-30OA- | |
|---|---|---|---|
| [ug/ml] | 1RIF | 1RIF | PT-50OA-1RIF |
| 0.05 | 4.12 | 4.39 | 1.67 |
| 0.1 | 8.33 | 11.32 | 2.11 |
| 0.5 | 12.54 | 21.4 | 4.65 |
| 1 | 24.12 | 43.33 | 10.35 |
| 2 | 29.12 | 47.46 | 13.95 |
| 3 | 44.04 | 62.11 | 18.33 |
| 5 | 51.14 | 71.4 | 19.56 |
| 10 | 60.35 | 73.95 | 20.93 |

### Ampicillin

Ampicillin is a beta lactam antibiotic which inhibits crosslinking of peptidoglycan leading to cell rupture. Table 17 shows the inhibition of P. aeruginosa by cubosome formulations containing the antibiotic Ampicillin, as presented in Table 16.

Between 0.5-10 μg/ml the encapsulated formulation displayed 50-70% more inhibition than the free antibiotic. For example, 62% compared to 39% at 0.5 μg/ml. The enhancement plateaus at higher concentrations due to time-dependence of the drug vs concentration dependence.

TABLE 16
Formulations containing ampicillin.

| | | | Avg | |
|---|---|---|---|---|
| | | Lattice Par. | curvature | |
| Formulation | Phase | (Å) | (nm −1) | Avg CPP |
| MO-10OA- Amp | Pn3m | 86.5 | −0.270 | 1.193 |

TABLE 17
Percentage inhibition of Pseudomonas aeruginosa by incubation
with MO-OA-Ampicillin cubosomes with varying concentrations.

| Concentration | | Free Ampicillin | |
|---|---|---|---|
| ug/ml | MO-10-OA-AMP | control ug/ml | % Inhibition |
| 0.5 | 61.49 | 0.5 | 38.64 |
| 5 | 70.58 | 2.5 | 39.83 |
| 10 | 75.04 | 5 | 41.85 |
| 20 | 78.38 | 7.5 | 42.81 |
| 40 | 85.76 | 10 | 46.37 |
| 60 | 92.79 | 15 | 62.19 |
| 80 | 98.96 | 20 | 69.92 |
| 100 | 99.73 | 25 | 72.29 |

### Vancomycin

Vancomycin is a glycopeptide antibiotic which inhibits cell wall synthesis and is typically inactive against gram-negative strains (e.g. P. aeruginosa) due to diffusion barriers across the membrane and altered targets.

Table 19 shows the inhibition of P. aeruginosa by cubosome formulations containing the antibiotic vancomycin, as presented in Table 18. At concentrations between 0.5-20 ug/ml, the encapsulated vancomycin yielded at least 2.7 times more inhibition. At the lowest concentration, 0.5 ug/ml, encapsulation yielded 16 times more inhibition. This indicates an advantage of the present approach in being able to effectively deliver and make use of drugs which may not typically be effective against gram-negative bacteria due to difficulties in diffusion across the bacterial membrane.

TABLE 18
Formulation of cubosomes containing vancomycin.

| | | | Avg | |
|---|---|---|---|---|
| | | Lattice Par. | curvature | |
| Formulation | Phase | (Å) | (nm −1) | Avg CPP |
| MO-Van | Im3m | 141.27 | −0.161 | 1.170 |
| MO-DOTAP-Van | Im3m | 144.29 | −0.1596 | 1.168 |
| MO-10OA-Van | Pm3m | 84.9 | −0.270 | 1.193 |

TABLE 19
Percentage inhibition of Pseudomonas aeruginosa
by incubation with MO-OA-vancomycin cubosomes with
varying concentrations. (Note: Literature MICs (90%
inhibition) highly variable, often 256 ug/ml++).

| Concentration | | Free concentration | |
|---|---|---|---|
| ug/ml | MO-10-OA-VANC | ug/ml | Inhibition % |
| 0.5 | 20.32 | 0.50 | 1.24 |
| 5 | 33.1 | 2.50 | 9.83 |
| 10 | 43.74 | 5.00 | 11.60 |
| 25 | 51.28 | 7.50 | 14.21 |
| 50 | 62.86 | 10.00 | 16.60 |
| 100 | 71.86 | 15.00 | 17.57 |
| 150 | 88.59 | 20.00 | 18.03 |
| 200 | 96.13 | 25.00 | 18.42 |

### Dual Drug Loading

The periodic three-dimensional topology of the lipid particles of the present disclosure facilitates the encapsulation of multiple drug cargoes within the same particle. This is demonstrated below for combinations of Novo-Fus, Rif-Fus and Pip-Fus.

TABLE 20
Inhibition of E. coli and P. aeruginosa by cubosomes encapsulating
novobiocin, versus novobiocin and fusidic acid.

| E. coli | | | | |
|---|---|---|---|---|
| | | MO-TAP- | P. aeruginosa | |
| | | 3Novo- | MO- | MO-DOTAP- |
| MO-DOTAP 3% | MO-3Novo-3FUS- | 3FUS- | DOTAP- | 3Novo-3FUS- |
| Novo-ECOLI | ECOLI | ECOLI | 3Novo-PS | PS |
| 11.37 | 13.02 | 22.23 | 22.75 | 30.43 |
| 19.33 | 21.98 | 27.70 | 25.82 | 40.67 |
| 24.39 | 36.83 | 33.27 | 29.52 | 41.24 |
| 32.35 | 50.11 | 43.06 | 35.60 | 47.89 |
| 38.91 | 57.99 | 52.68 | 42.32 | 60.01 |
| 43.55 | 72.59 | 71.51 | 47.63 | 64.90 |
| | 88.93 | 83.21 | 55.51 | 84.92 |
| | 98.81 | 94.41 | 68.60 | 92.83 |

TABLE 21
Inhibition of E. coli by cubosomes encapsulating
novobiocin, versus novobiocin and fusidic acid.

| MO-3Rif-3fus | MO-DOTAP- 3% Rif 3% fus |
|---|---|
| 23.83 | 28.05 |
| 27.97 | 31.66 |
| 50.3 | 39.13 |
| 52.06 | 45.91 |
| 54 | 53.03 |
| 55.4 | 60.24 |
| 63.23 | 66.66 |
| 70.27 | 72.82 |

TABLE 22
Inhibition of P. aeruginosa by cubosomes encapsulating
piperacillin, versus piperacillin and fusidic acid.

| MO-3PIP- | MO-3 Pip-3Fus |
|---|---|
| 6.86 | 3.86 |
| 20.75 | 6.08 |
| 28.47 | 16.32 |
| 35.08 | 20.19 |
| 42.88 | 30.03 |
| 48.11 | 37.03 |
| 53.51 | 44.25 |
| 54.54 | 70.08 |

### Fusidic Acid

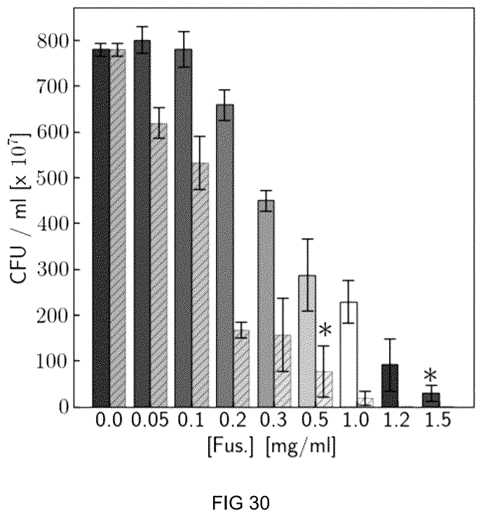

Fusidic acid is a bacteriostatic which interferes with protein synthesis. It is exceedingly hydrophobic and consequently has minimal utility against gram-negative bacteria. When employed in the lipid particles of the present disclosure, a significant drop was observed in E. coli colony forming units (CFU), particularly as the concentration increased as seen in FIG. 30. A marked drop in CFU count was observed at Fus concentrations beyond 0.1 mg/ml and was reduced by more than half at 0.2 mg/ml. The respective minimum inhibitory concentrations (MICs), noted by * on the plot, were 0.5 mg/ml and 1.5 mg/ml for the encapsulated and free drug, respectively. MIC references to the minimum concentration to achieve 90% inhibition of cell proliferation. Again, it is noted that Fus typically has limited activity against gram-negative bacteria due to poor transport across the outer membrane due to the molecular size and hydrophobicity.

### Controls

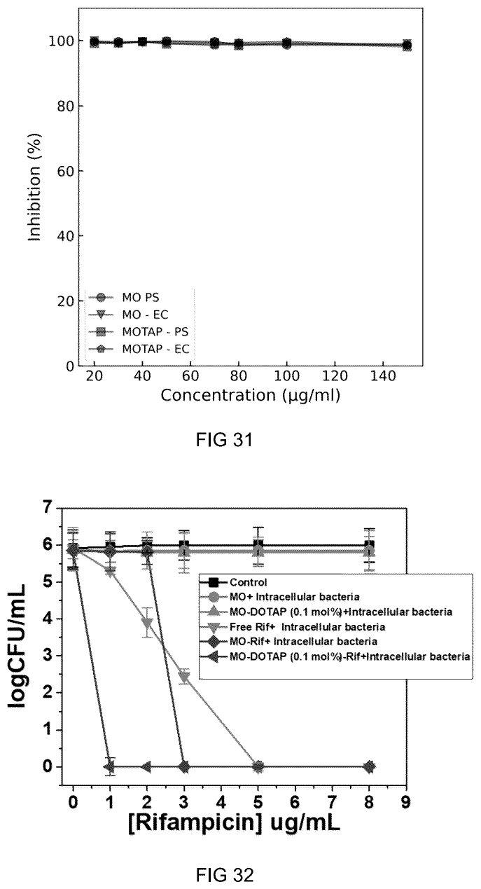

The toxicity of the nanoparticles without loaded active agent was examined against E. coli and P. aeruginosa. From the results shown in FIG. 31, the lipid cubosome particles have negligible impact on the bacteria viability when no antibiotics are encapsulated.

### Formulation of Anti-TB Drugs Loaded MO-Based Cubosome

Commercially available TB drugs have a short life and rapid clearance, which limits their efficacy. To increase the efficacy of anti-tubercular drugs and improve success rates for tuberculosis treatment, an effective and robust delivery system is required, such as the lipid particle delivery of the present disclosure.

The present experiment evaluated the potential of monoolein (MO) based cubosomes for the encapsulation and delivery of single anti-tubercular drugs (rifampicin, ethambutol, isoniazid, pyrazinamide and streptomycin) against Mycobacterium smegmatis and Mycobacterium tuberculosis H37Ra in-vitro culture model.

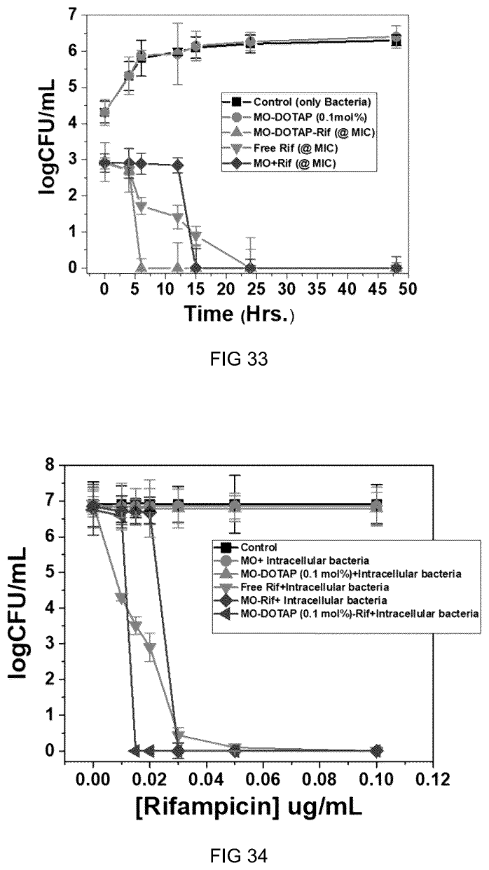

To test this, five anti-TB drugs, rifampicin, isoniazid, pyrazinamide, ethambutol, and streptomycin were encapsulated in MO cubosomes. Encapsulation of the hydrophobic drugs rifampicin and isoniazid (which have octanol-water partition coefficients of 4.24 and −0.70 respectively) resulted in an increase in lattice parameter of the Im3m phase. Drug loading of up to 4 mol % was achieved for rifampicin and 15 mol % of isoniazid. The results are shown in FIG. 32 to 35 wherein FIG. 32 indicates the MIC determination for Mycobacterium smegmatis and FIG. 33 indicates the cell death at MIC for Mycobacterium smegmatis. The reported MIC value of Rif for intracellular bacteria is 4-5 μg/mL and it was found the MIC value in the presence of MO-Rif (1 mol %) for intracellular bacteria is 3 μg/mL while the MIC value in the presence of MO-DOTAP (0.1 mol %) Rif for intracellular bacteria is 1 μg/mL. The reported value for cell death is around 20-24 hours. The MO-Rif reduced the incubation time @MIC value from 24 hours to 15 hours while MO-DOTAP (0.1 mol %)-Rif (1 mol %) reduced the incubation time @MIC value from 24 hours to 6 hours.

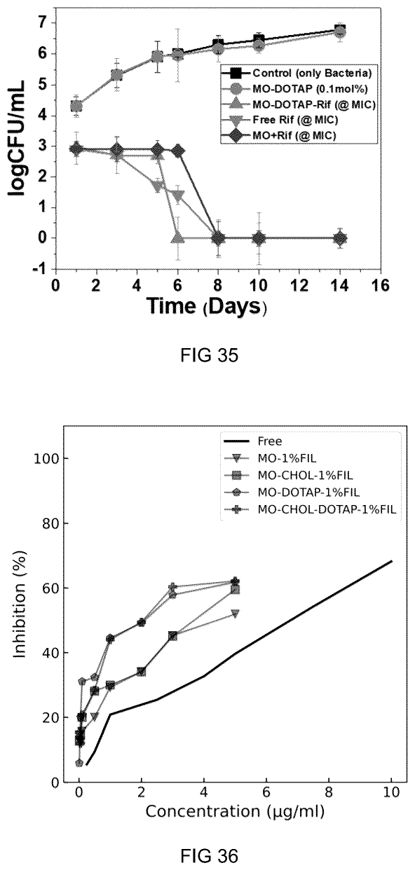

FIG. 34 indicates the MIC determination for Mycobacterium tuberculosis H37Ra and FIG. 35 indicates the cell death at MIC for Mycobacterium tuberculosis H37Ra. The reported MIC value of Rif for intracellular bacteria is 0.04-0.05 μg/mL and it was found the MIC value in the presence of MO-Rif (1 mol %) for intracellular bacteria is 0.03 μg/mL while the MIC value in the presence of MO-DOTAP (0.1 mol %) Rif for intracellular bacteria is 0.015 μg/mL. The reported value for cell death at MIC is around 8-9 days. The MO-Rif reduced the incubation time @MIC value to 7-8 days while MO-DOTAP (0.1 mol %)-Rif (1 mol %) reduced the incubation time @MIC value from 8 days to 5 days.

Rifampicin encapsulated in the cubosomes therefore had a significantly greater killing effect as compared to the free drug and has the potential to shorten the lifecycle duration of axenic bacterial culture. Furthermore, incorporation of positive charge by addition of cationic lipid DOTAP (0.1 mol %) significantly increased the killing effect. Encapsulation of the anti-TB drug rifampicin in cationic cubosomes improved the drug bioavailability in in vitro studies, with the time taken to eliminate the bacilli burden reduced from three to one day. This could reduce the dosage frequency and potentially resolve the difficulty of poor patient compliance due to prolonged TB drug treatment regimes.

TABLE 23
The phase adopted, lattice parameter and size of MO cubosomes
following encapsulation of rifampicin (A), isoniazid (B),
ethambutol (C), streptomycin (D) and pyrazinamide (E).

| | | | |
|---|---|---|---|
| Lipid | | | DLS |
| materials | LP (Å) | Phase | (nm) |

A

| | | | |
|---|---|---|---|
| MO | 143.02 | Im3m | 260 |
| MO - (1 mo %) | 149.27 | Im3m | 336 |
| Rifampicin | | | |
| MO - (2 mo %) | 156.34 | Im3m | 344 |
| Rifampicin | | | |
| MO - (3 mo %) | 182.79 | Im3m | 395 |
| Rifampicin | | | |
| MO - (4 mo %) | 185.59 | Im3m | 595 |
| Rifampicin | | | |
| MO - (5 mo %) | 46.76 | Lα | 627 |
| Rifampicin | | | |

B

| | | | |
|---|---|---|---|
| MO | 140.00 | Im3m | |
| MO - (1 mo %) | 134.50 | Im3m | 244 |
| Isoniazid | | | |
| MO - (5 mo %) | 146.72 | Im3m | 256 |
| Isoniazid | | | |
| MO - (10 mo %) | 146.28 | Im3m | 285 |
| Isoniazid | | | |
| MO - (15 mo %) | 150.52 | Im3m | 347 |
| Isoniazid | | | |
| MO - (20 mo %) | 180.51 | Im3m | 373 |
| Isoniazid | | | |

C

| | | | |
|---|---|---|---|
| MO | 140.32 | Im3m | |
| MO - (1 mo %) | 139.57 | Im3m | 307 |
| Ethambutol | | | |
| MO - (5 mo %) | 140.58 | Im3m | 387 |
| Ethambutol | | | |
| MO - (10 mo %) | 140.73 | Im3m | 469 |
| Ethambutol | | | |
| MO - (15 mo %) | 141.10 | Im3m | 576 |
| Ethambutol | | | |
| MO - (20 mo %) | 141.79 | Im3m | 505 |
| Ethambutol | | | |

D

| | | | |
|---|---|---|---|
| MO | 140.23 | Im3m | |
| MO - (1 mo %) | 147.83 | Im3m | 328 |
| Streptomycin | | | |
| MO - (5 mo %) | 143.65 | Im3m | 311 |
| Streptomycin | | | |
| MO - (10 mo %) | 138.22 | Im3m | 208 |
| Streptomycin | | | |
| MO - (15 mo %) | 140.27 | Im3m | 217 |
| Streptomycin | | | |
| MO - (20 mo %) | 140.95 | Im3m | 177 |
| Streptomycin | | | |

E

| | | | |
|---|---|---|---|
| MO | 140.02 | Im3m | |
| MO - (1 mo %) | 135.49 | Im3m | 231 |
| Pyrazinmid | | | |
| MO - (5 mo %) | 136.69 | Im3m | 319 |
| Pyrazinmid | | | |
| MO - (10 mo %) | 134.58 | Im3m | 256 |
| Pyrazinmid | | | |
| MO - (15 mo %) | 134.33 | Im3m | 251 |
| Pyrazinmid | | | |
| MO - (20 mo %) | 134.63 | Im3m | 271 |
| Pyrazinmid | | | |

TABLE 24
The phase adopted, lattice parameter and size of
MO cubosomes following incorporation of DOTAP.

| | Lattice | | Size | ζ-potential |
|---|---|---|---|---|
| Lipid materials | parameter (LP) | Phase | (nm) | (mV) |
| MO | 142.3 | Im3m | 215.6 | 0.3 |
| MO-DOTAP (0.1 mol %)- | 150.1 | Im3m | 275.2 | 28.6 ± 0.3 |
| Rif (0 mol %) | | | | |
| MO-DOTAP (0.1 Mol %)- | 155.9 | Im3m | 321.5 | 25.1 ± 0.2 |
| Rif (1 mol %) | | | | |
| MO-DOTAP (0.1 Mol %)- | 186.7 | Im3m | 332.4 | 25.3 ± 0.1 |
| Rif (2 mol %) | | | | |
| MO-DOTAP (0.1 Mol %)- | 188.6 | Im3m | 338.1 | 25.4 ± 0.3 |
| Rif (3 mol %) | | | | |
| MO-DOTAP (0.1 Mol %)- | 46.6 | L_{α} | 341.2 | 26.6 ± 0.4 |
| Rif (4 mol %) | | | | |
| MO-DOTAP (0.1 Mol %)- | 47.7 | L_{α} | 352.1 | 27.8 ± 0.3 |
| Rif (5 mol %) | | | | |

Interaction with Fungi

### Filipin and Amphotericin B

The efficacy of antifungals was examined against the fungal strain Candida albicans. Filipin and amphotericin B are antifungals which act by removing ergosterol from the plasma membrane which prompts cell leakage and death. The compounds are both hydrophobic, with some lipid-complex products available. All encapsulation formulations of the present disclosure for filipin yielded increased inhibition over the free drug (FIG. 36). The incorporation of positive charge by inclusion of cationic lipid DOTAP greatly increased the inhibition. This can be observed by the groupings of particles with cationic properties (plus and pentagon symbol) and those without (triangle and square).

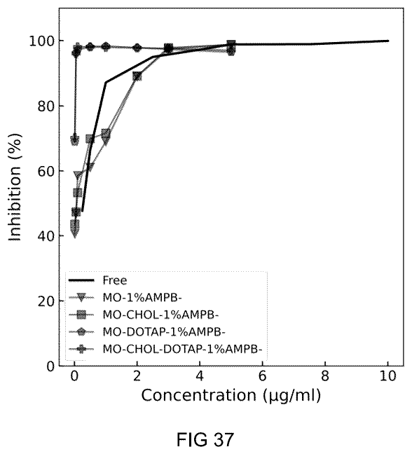

All encapsulation formulations for amphotericin yielded inhibition at least equivalent to the freely dissolved drug (FIG. 37). Inclusion of cationic lipids again greatly increased the inhibition. Compared to the freely dissolved drug, the concentration required to achieve 98% inhibition reduced by a factor of 100, from 10 ug/ml to 0.1 ug/ml.

TABLE 25
Formulation of cubosomes containing 1% filipin,
0/1% DOTAP (positive charge), 0/10% cholesterol.

| | | | Avg | |
|---|---|---|---|---|
| | | Lattice Par. | curvature | |
| Formulation | Phase | (Å) | (nm −1) | Avg CPP |
| MO-FIL | Im3m | 153.7 | −0.161 | 1.170 |
| MO-CHOL-FIL | Im3m | 148 | −0.189 | 1.173 |
| MO-DOTAP-FIL | Im3m | 162.7 | −0.159 | 1.168 |
| MO-CHOL- | Im3m | 160.1 | −0.187 | 1.171 |
| DOTAP-FIL | | | | |

TABLE 26
Formulations of cubosomes containing 1% amphotericin
B, 0/1% DOTAP (positive charge), 0/10% cholesterol.

| | | | Avg | |
|---|---|---|---|---|
| | | Lattice Par. | curvature | |
| Formulation | Phase | (Å) | (nm −1) | Avg CPP |
| MO-AmpB | Im3m | 137 | −0.161 | 1.170 |
| MO-CHOL-AmpB | Im3m | 146.4 | −0.189 | 1.173 |
| MO-DOTAP- | Pn3m | 143.1 | −0.159 | 1.168 |
| AmpB | | | | |
| MO-CHOL- | Im3m | 157.5 | −0.187 | 1.171 |
| DOTAP-AmpB | | | | |

### Fluconazole

Fluconazole controls fungal diseases by impairing ergosterol synthesis, an essential structural component of the fungal cell membrane. However, systemic therapy with fluconazole poses two significant problems being drug toxicity and drug resistance.

Synthesis of ionisable aminolipid: The aminolipid Morpholine oley ester (MOE/Lipid-5) was synthesised using an esterification reaction between 4-(2-Hydroxyethyl) morpholine and Oleic acid (OA). In brief, amino alcohol (1.1 eq) was added to the cooled solution of OA (1.0 eq) in DCM, EDCl (1.1 eq) and DMAP (0.2 eq). The reaction temperature was stirred at RT for 24 hours. The solvent was removed using a rotary evaporator, and a flash silica column purified the obtained crude material. The synthesised lipid structure was confirmed by nuclear magnetic resonance (NMR) imaging (11H) analysis. Lipid nanoparticles with fluconazole and empty nanoparticles without fluconazole were prepared by a dry lipid hydration method. A dry film was prepared by dissolving MO (20 mg/1 mL of ethanol) and MOE (20 mg/1 mL of ethanol) and mixing them in a ratio of 70:30 and removing ethanol by keeping it in a vacuum oven for 12 hours. Empty nanoparticles without the drug were prepared by hydrating the formed dry lipid with 1 mL of F127 (2 mg in 1 mL of DI water) solution. The resulting mixture was sonicated in pulse mode for 5 min using a probe sonicator (Q Sonica) to obtain opaque dispersion. Fluconazole encapsulated formulations were prepared by hydrating the formed dry lipid with 1 mL of DI water containing F127 and Fluconazole (2 mg each). The resulting mixture was sonicated in pulse mode for 5 min using a probe sonicator (Q Sonica) to obtain an opaque dispersion.

Stains and growth conditions: the fungi fluconazole-resistant Cryptococcus neoformans was obtained from SA Pathology and stored in potato dextrose (PD) broth at −80° C. The fungi were cultured overnight in potato dextrose (PD) plates at 37° C. before use. An inoculation loop was used to suspend cells in PDB broth, and the optical density was adjusted to ˜2 at 600 nm (OD600) using a UV-vis spectrometer. Fungal suspension of 5 μl will be added to a final volume of 100 μl of a 96-well plate to obtain a final OD600 concentration of ˜0.1.

Antimicrobial Assays: The minimum inhibitory concentration of the antimicrobials being the fluconazole free agent, control nanoparticles without the drug, and fluconazole encapsulated lipid nanoparticles to inhibit the growth of 50% (MIC-50), and 90% (MIC-90) of the fluconazole-resistant C. neoformans was investigated using a 96 well plate. The 96-well plate was incubated for 24 hours in a UV-vis spectrophotometer, and spectra were collected in the range of 220-1000 nm. In summary, the plate contained a range of concentrations of the antimicrobials incubated with the fungi, including a fungi control, a PD broth control, and antimicrobial controls. This experiment was conducted at pH 5.2 and pH 7.1.

### Results

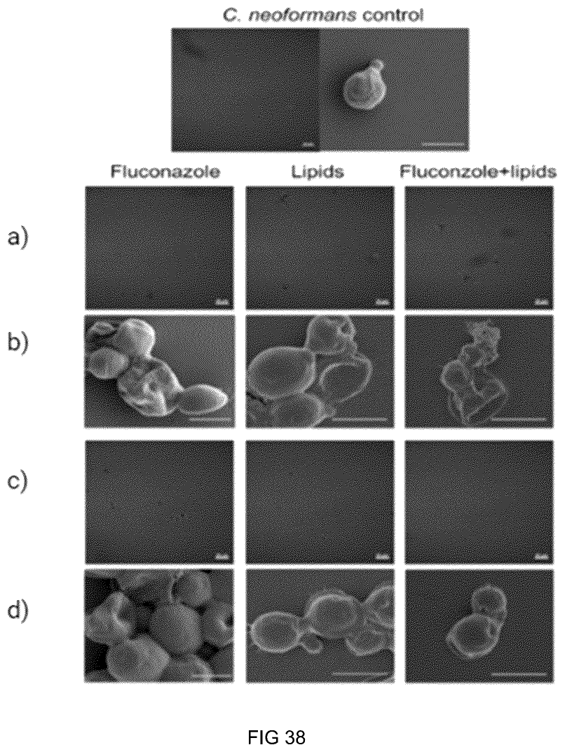

O2ME is neutral at pH-7.4 and positively charged at lower pH (4-6.0) and will cause head group expansion and charge repulsion at lower pH. Incorporating 30 wt % of O2ME to MO at pH-7.4 resulted in hexosomes with an internal hexagonal phase. Due to head group expansion and charge repulsion at lower pH values, the membrane curvature decreases and changes mesophase from hexagonal to cubic. Due to the fluconazole resistance, treating the infection with fluconazole alone fails to reach MIC-90, but the encapsulated formulation with cubic structure and the positive charge has a MIC-90 value of 123.015 μg/ml at pH 5.17 compared to 183.843 ug/ml at pH 7.08. The SEM and confocal images support the disruption of the fungal wall as seen in FIG. 38.

TABLE 27
The minimum inhibitory concentration of the fluconazole loaded
nanoparticle compared to fluconazole alone and control nanoparticles
without the drug to inhibit the growth of 50% (MIC-50) and 90% (MIC-
90) of the fluconazole-resistant C. neoformans.

| | Based on % inhibition | pH 5.17 | pH 7.08 |
|---|---|---|---|
| MIC-50 | Control nanoparticle | 1806 μg/ml | 4172 μg/ml |
| | without fluconazole | | |
| | Fluconazole only | 12.359 μg/ml | 12.174 μg/ml |
| | Fluconazole | 15.926 μg/ml | 23.989 μg/ml |
| | encapsulated | | |
| | nanoparticle | | |
| MIC-90 | Control nanoparticle | 1962 μg/ml | 9976 μg/ml |
| | without fluconazole | | |
| | Fluconazole only | ND | ND |
| | Lipid-encapsulated | 123.015 μg/ml | 183.843 μg/ml |
| | fluconazole | | |
| | particles | | |

*ND denotes not determined as 90% inhibition was not achieved

TABLE 28
The internal mesophases of the fluconazole loaded
nanoparticles at different pH values.

| | Mesophase observed at different pH at 37° C. | | | | | | | | | | |
|---|---|---|---|---|---|---|---|---|---|---|---|
| | | | | | | | | | | Cell | Cellmedia+ |
| Nanoparticle | 4.0 | 4.5 | 5.0 | 5.5 | 6.0 | 6.5 | 7.0 | 7.5 | 8 | media | FBS |
| Fluconazole | N/S | N/S | Im3m | Pn3m | H₂ | H₂ | H₂ | H₂ | H₂ | H₂ | H₂ |
| loaded | | | | | | | | | | | |
| nanoparticle | | | | | | | | | | | |

*N/S denotes a non-scattered peak; this may be an emulsion.

### REFERENCE LIST

1. Santos, R. S., Figueiredo, C., Azevedo, N. F., Braeckmans, K. & De Smedt, S. C. Nanomaterials and molecular transporters to overcome the bacterial envelope barrier: Towards advanced delivery of antibiotics. Adv. Drug Deliv. Rev. 136, 28-48 (2018).
2. Sahay, G. et al. Efficiency of siRNA delivery by lipid nanoparticles is limited by endocytic recycling. Nat. Biotechnol. 31, 653 (2013).
3. Oh, N. & Park, J.-H. Endocytosis and exocytosis of nanoparticles in mammalian cells. Int. J. Nanomedicine 9, 51 (2014).
4. Deshpande, S. & Singh, N. Influence of Cubosome Surface Architecture on Its Cellular Uptake Mechanism. Langmuir 33, 3509-3516 (2017).
5. Prange, J. A. et al. Overcoming Endocytosis Deficiency by Cubosome Nanocarriers. ACS Appl. Bio Mater. (2019).
6. Vandoolaeghe, P., Barauskas, J., Johnsson, M., Tiberg, F. & Nylander, T. Interaction between lamellar (vesicles) and nonlamellar lipid liquid-crystalline nanoparticles as studied by time-resolved small-angle X-ray diffraction. Langmuir 25, 3999-4008 (2009).
7. Chang, D. P. et al. Non-lamellar lipid liquid crystalline structures at interfaces. Adv. Colloid Interface Sci. 222, 135-147 (2015).
8. Thorn, C. R., Clulow, A. J., Boyd, B. J., Prestidge, C. A. & Thomas, N. Bacterial lipase triggers the release of antibiotics from digestible liquid crystal nanoparticles. J. Control. Release (2019).

1. A method of treatment or prevention of a disease, disorder or condition associated with a gram-negative bacteria or a fungi, including the step of administering a therapeutically effective amount of a non-lamellar lyotropic liquid crystalline phase particle comprising an antibacterial or antifungal agent, the non-lamellar lyotropic liquid crystalline phase particle comprising one or more fusogenic amphiphilic lipids, to a subject in need thereof to thereby treat or prevent the disease, disorder or condition.

2. The method of claim 1 wherein the disease, disorder or condition is an infection caused by a gram-negative bacteria or a fungi.

3. The method of claim 1 wherein the non-lamellar lyotropic liquid crystalline phase particle encapsulates the antibacterial or antifungal agent within its channels or folds.

4. The method of claim 1 wherein the non-lamellar lyotropic liquid crystalline phase particle is formed by the self-assembly of the one or more fusogenic amphiphilic lipids.

5. The method of claim 1 wherein the non-lamellar lyotropic liquid crystalline phase particle comprises at least two fusogenic amphiphilic lipids.

6. (canceled)

7. The method of claim 1 wherein the non-lamellar lyotropic liquid crystalline phase particle has a bulk phase selected from the group consisting of the cubic phase, the hexagonal phase and the sponge phase.

8. The method of claim 1 wherein the non-lamellar lyotropic liquid crystalline phase particle is a cubosome.

9. The method of claim 1 wherein the non-lamellar lyotropic liquid crystalline phase particle has an internal curvature induced splay ( [symbol] ) of less than −0.05 nm⁻¹.

10. (canceled)

11. The method of claim 1 wherein the non-lamellar lyotropic liquid crystalline phase particle has a particle diameter of greater than about 50 nm.

12. The method of claim 1 wherein the non-lamellar lyotropic liquid crystalline phase particle comprises at least one stabiliser present at between 6 to 18 wt % of the particle.

13. The method of claim 1 wherein the non-lamellar lyotropic liquid crystalline phase particle comprises one or more positively charged lipids in an amount between 0.1 to less than 20 mol %.

14. The method of claim 1 wherein the non-lamellar lyotropic liquid crystalline phase particle further comprises a non-amphiphilic and/or non-fusogenic charged compound.

15. The method of claim 1 wherein the one or more fusogenic amphiphilic lipids are selected from those presenting a hydrophobic tail group selected from the group consisting of oleoyl, linoleoyl, linolenoyl, phytanoyl, farnesoyl and extended aliphatic hydrophobic.

16. The method of claim 1 wherein the one or more fusogenic amphiphilic lipids are selected from those presenting a headgroup selected from the group consisting of alcohol, carboxyl, poly-ol, sugar, amide, amine, lactate, glyceryl, diglyceryl, coordination complex, caprolactam, ether, acetate, quinone, and combinations thereof.

17. The method of claim 1 wherein the one or more fusogenic amphiphilic lipids are selected from the group consisting of 1-monoolein, 2-monoolein, citrem, oleoyl lactate, oleamide, monoelaidin, linoleic acid, elaidic acid, monopalmitolein, monolinolein, phytantriol, diolein, triolein, dioleoyl-glycerol, didodecyldimethylammonium bromide, dioctadecyl (dimethyl) ammonium chloride (DOAC/DODMAC) or bromide (DODAB), 1,2-dioleoyl-sn-glycero-3-phosphoethanolamine (DOPE), 1,2-Dioleoyl-phosphatidylglycerol (DOPG), oleic acid, lysol-hydroxy-2-oleoyl-sn-glycero-3-phosphocholine, 1,2-dioleoyl-sn-glycero-3-dihexyl-phosphocholine, vitamin E tocopherol, vitamin E (tocopheryl) acetate, phytanoyl monoethanolamide, farnesoyl monoethanolamide, oleoyl monoethanolamide, linoleoyl monoethanolamide and linolenoyl monoethanolamide.

18. The method of claim 5 wherein when the non-lamellar lyotropic liquid crystalline phase particle comprises at least two fusogenic amphiphilic lipids, then at least one of the fusogenic amphiphilic lipids is selected from monoolein and phytantriol.

19. The method of claim 1 wherein the antibacterial agent is a gram-negative bacteria antibacterial agent.

20. (canceled)

21. The method of claim 1 wherein the antibacterial or antifungal agent is hydrophobic.

22. (canceled)

23. (canceled)

24. The method of claim 1 wherein the antibacterial or antifungal agent is present at between 0.1 to 30.0 mol % of the particle.

25.-27. (canceled)

28. The method of claim 1 wherein the non-lamellar lyotropic liquid crystalline phase particle comprises a monoolein.
